(19) United States
(12) Patent Application Publication
(10) Pub. No.: US 2026/0193396 A1
(43) Pub. Date: Jul. 9, 2026

(54) NOVEL COPOLYMERS; PROCESS FOR PREPARING SAME; COSMETIC USE THEREOF; COSMETIC FORMULATIONS COMPRISING SAME

(71) Applicant: SOCIETE D'EXPLOITATION DE PRODUITS POUR LES INDUSTRIES CHIMIQUES SEPPIC, Paris (FR)

(72) Inventors: Stephane Monteillet, Paris (FR); Miruna Bodoc, Paris (FR)

(73) Assignee: SOCIETE D'EXPLOITATION DE PRODUITS POUR LES INDUSTRIES CHIMIQUES SEPPIC, Paris (FR)

(21) Appl. No.: 19/442,696

(22) Filed: Jan. 7, 2026

(30) Foreign Application Priority Data
Jan. 8, 2025 (FR) 2500147

(51) Int. Cl. C08F 224/00; A61K 8/81; A61Q 17/04; C08F 2/10; C08K 5/14; C08K 5/29

(52) U.S. Cl. CPC C08F 224/00; A61K 8/8164; A61Q 17/04; C08F 2/10; C08K 5/14; C08K 5/29; A61K 2800/48; A61K 2800/52

(57) ABSTRACT

Linear or crosslinked copolymers consist of monomer units derived from Tulipalin A and from itaconic acid are present at a molar ratio RA, having as numerator a number of moles of monomer units derived from the compound of formula (I) and as denominator a number of moles of monomer units derived from the compound of formula (II), that is greater than or equal to 0.4 and less than or equal to 3.0. A process for preparing the linear or crosslinked polymer, its use in dermocosmetics or dermopharmacy, and to topical formulations including the linear or crosslinked polymer are also described.

### TECHNICAL FIELD OF THE INVENTION

The invention falls within the field of the cosmetic and pharmaceutical industries. The invention relates more particularly to novel thickening agents of natural origin.

### PRIOR ART

Polymers are widely used these days in topical formulations for use in the fields of cosmetics, dermocosmetics, pharmacy or dermopharmacy. Most of them are rheology modifiers, which thicken polar phases, such as water. Aqueous-phase-thickening synthetic polymers include linear or crosslinked polyelectrolytes which have the ability to be deployed in a polar solvent owing to their affinity with these phases and because of the electrostatic repulsions due to the presence of charges on their polymer backbone. This deployment in polar phases, creating a polymer network, results in an increase in viscosity and the provision of consistency and stabilization in formulations containing oils, of gel-cream or emulsion type.

Examples of such polymers include crosslinked polyelectrolytes, which have the ability to be deployed in a polar solvent in particular because of the electrostatic repulsive forces due to the presence of electric charges on their polymer backbone. This phenomenon thus makes it possible to form a polymer network which leads to an increase in viscosity, a consistency and a stabilization of formulations such as oil-in-water or water-in-oil emulsions in the presence or absence of emulsifying surfactants or gel-creams free of emulsifying surfactants. These polymers generally result from the polymerization of acrylate or methacrylate monomers or monomers derived from acrylamide or its derivatives.

Examples of crosslinked polyelectrolytes sold on the market or disclosed in the literature include:
The homopolymer of acrylic acid partially or completely salified with a sodium, ammonium or potassium salt, the homopolymer of methacrylic acid partially or completely salified with a sodium, ammonium or potassium salt, the homopolymer of 2-methyl-((1-oxo-2-propenyl)amino)-1-propanesulfonic acid (AMPS) partially or completely salified with a sodium salt, ammonium or potassium salt,
copolymers of acrylic acid, partially or completely salified with a sodium, ammonium or potassium salt and of AMPS, copolymers of methacrylic acid, partially or completely salified with a sodium, ammonium or potassium salt and of AMPS,
copolymers of acrylamide and of AMPS, partially or completely salified with a sodium, ammonium or potassium salt,
copolymers of vinylpyrolidone and of AMPS, partially or completely salified with a sodium, ammonium or potassium salt,
copolymers of AMPS, partially or completely salified with a sodium, ammonium or potassium salt, and of (2-hydroxyethyl) acrylate or (2-hydroxyethyl) methacrylate, or (2,3-dihydroxypropyl) acrylate or (2,3-dihydroxypropyl) methacrylate,
copolymers of AMPS, partially or completely salified with a sodium, ammonium or potassium salt, and of hydroxyethylacrylamide, or hydroxyethylmethacrylamide,
copolymers of AMPS, partially or completely salified with a sodium, ammonium or potassium salt, and of N,N-dimethylacrylamide, N,N-dimethylmethacrylamide, N,N-diethylacrylamide, N,N-diethylmethacrylamide, N-methylacrylamide, N-ethylacrylamide, N-propylacrylamide, N-isopropylacrylamide, N-butylacrylamide, N-(tert-butyl)acrylamide, N-methylmethacrylamide, N-ethylmethacrylamide, N-propylmethacrylamide, N-isopropylmethacrylamide, N-butylmethacrylamide, N-(tert-butyl)methacrylamide or N,N-dipropylacrylamide,
copolymers of AMPS, partially or completely salified with a sodium, ammonium or potassium salt, and of tris(hydroxymethyl)acrylamidomethane (THAM),
copolymers of acrylic acid or methacrylic acid, partially or completely salified with a sodium, ammonium or potassium salt, and of (2-hydroxyethyl) acrylate, or (2,3-dihydroxypropyl) acrylate,
copolymers of acrylic acid or methacrylic acid, partially or completely salified with a sodium, ammonium or potassium salt, and of (2-hydroxyethyl) methacrylate, or (2,3-dihydroxypropyl) methacrylate,
copolymers of acrylic acid or methacrylic acid, partially or completely salified with a sodium, ammonium or potassium salt, and of hydroxyethylacrylamide,
copolymers of acrylic acid or methacrylic acid, partially or completely salified with a sodium, ammonium or potassium salt, and of THAM,
copolymers of acrylic acid or methacrylic acid, partially or completely salified with a sodium, ammonium or potassium salt, and of N,N-dimethylacrylamide, N,N-dimethylmethacrylamide, N,N-diethylacrylamide, N,N-diethylmethacrylamide, N-methylacrylamide, N-ethylacrylamide, N-propylacrylamide, N-isopropylacrylamide, N-butylacrylamide, N-(tert-butyl)acrylamide, N-methylmethacrylamide, N-ethylmethacrylamide, N-propylmethacrylamide, N-isopropylmethacrylamide, N-butylmethacrylamide, N-(tert-butyl)methacrylamide or N,N-dipropylacrylamide,
branched or crosslinked terpolymers of acrylic acid or methacrylic acid, partially or completely salified with a sodium, ammonium or potassium salt, of AMPS, partially or completely salified with a sodium, ammonium or potassium salt, and of (2-hydroxyethyl) acrylate or (2-hydroxyethyl) methacrylate, or (2,3-dihydroxypropyl) acrylate or (2,3-dihydroxypropyl) methacrylate,
branched or crosslinked terpolymers of acrylic acid or methacrylic acid, partially or completely salified with a sodium, ammonium or potassium salt, of AMPS, partially or completely salified with a sodium, ammonium or potassium salt, and of THAM,
branched or crosslinked terpolymers of acrylic acid or methacrylic acid, partially or completely salified with a sodium, ammonium or potassium salt, of AMPS, partially or completely salified with a sodium, ammonium or potassium salt, and of N,N-dimethylacrylamide, N,N-dimethylmethacrylamide, N,N-diethylacrylamide, N,N-diethylmethacrylamide, N-methylacrylamide, N-ethylacrylamide, N-propylacrylamide, N-isopropylacrylamide, N-butylacrylamide, N-(tert-butyl)acrylamide, N-methylmethacrylamide, N-ethylmethacrylamide, N-propylmethacrylamide, N-isopropylmethacrylamide, N-butylmethacrylamide, N-(tert-butyl)methacrylamide or N,N-dipropylacrylamide,
branched or crosslinked terpolymers of acrylic acid or methacrylic acid, partially or completely salified with a sodium, ammonium or potassium salt, of AMPS, partially or completely salified with a sodium, ammonium or potassium salt, and of acrylamide or methacrylamide,
copolymers of acrylic acid or methacrylic acid, partially or completely salified with a sodium, ammonium or potassium salt, and of alkyl acrylates or alkyl methacrylate in which the carbon chain comprises between four and thirty carbon atoms and more particularly between ten and thirty carbon atoms, and
copolymers of AMPS, partially or completely salified with a sodium, ammonium or potassium salt, and of alkyl acrylates or alkyl methacrylate in which the carbon chain comprises between four and thirty carbon atoms and more particularly between ten and thirty carbon atoms.

Nowadays, these polymers, whether they are in the form of self-invertible inverse latices, concentrated inverse latices, or powders, make it possible to meet the customers' needs in terms of thickening performance, in a polar solvent, such as water. The aqueous gels obtained by dispersing them in this solvent have a smooth appearance, free from grains or lumps, with specific sensory properties to the touch, and also an ease of handling and of applying on the skin.

Despite the large commercial offering, the demand for new thickeners for aqueous phases for the formulation of cosmetic and dermopharmaceutical products remains current because the polymers used today are predominantly of petrochemical origin, while the user industries are engaged in an ecodesign process. It is therefore necessary to develop alternative thickeners which, while having comparable properties, have an improved environmental profile, either due to their origin or that of their precursors, or due to their biodegradable nature.

It is known to those skilled in the art that hydrocolloids, polysaccharides or proteins may have thickening, gelling and stabilizing properties. But very often, these natural products have unsatisfactory sensory properties, giving the cosmetic formulations a tacky, runny and soapy feel.

The inventors have therefore sought to develop new thickening polymers that are synthesized from monomers of natural origin and that give the cosmetic or pharmaceutical topical formulations with a sensory profile accepted by the end user.

### DESCRIPTION OF THE INVENTION

This is the reason why, according to a first aspect, the invention relates to a linear or crosslinked copolymer consisting of monomer units derived from 3-methylenedihydro-2(3H)-furanone of formula (I):

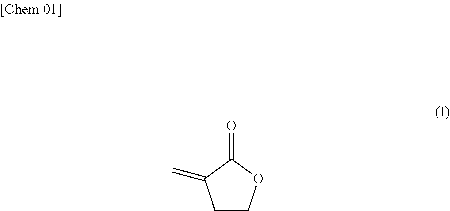

and of monomer units derived from methylene-1,4-butanedioic acid of formula (II):

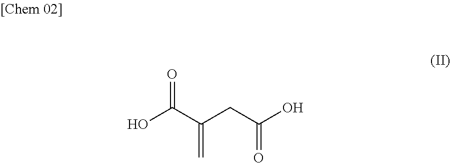

wherein the molar ratio RA, having as numerator the number of moles of monomer units derived from the compound of formula (I) and as denominator the number of moles of monomer units derived from the compound of formula (II), is greater than or equal to 0.4 and less than or equal to 3.0.

In the context of the present invention, the monomers of formula (I) and of formula (II) as defined above are commercially available and can be obtained from biomass. α-Methylene-γ-butyrolactone is known as Tulipalin A and methylene-1,4-butanedioic acid of formula (II) is known as itaconic acid.

According to a particular aspect of the present invention, the linear or crosslinked copolymer as defined above is characterized in that said molar ratio RA is greater than or equal to 1.5 and less than or equal to 2.5.

According to another particular aspect of the present invention, when the copolymer as defined above is crosslinked, the molar ratio RB, having as numerator the number of moles of monomer units derived from the crosslinking monomer and as denominator the sum of the number of moles of monomer units derived from the compound of formula (I) and of moles of monomer units derived from the compound of formula (II), is greater than or equal to 0.0010 and less than or equal to 0.0070 and is more particularly greater than or equal to 0.0020 and less than or equal to 0.0060.

According to another particular aspect of the present invention, when the copolymer as defined above is crosslinked, the monomer units derived from the crosslinking monomer are from a compound chosen from the group consisting of methylene bis(acrylamide) (MBA) (CAS number=110-26-9), pentaerythritol tetraacrylate (PETA) (CAS number=4986-89-4), trimethylolpropane triacrylate (TMPTA) (CAS number=15625-89-5), triallylamine (TAA), pentaerythritol triallyl ether (APE) (CAS number=1471-17-6), 1,6-hexanediol diacrylate (HDDA) (CAS number=13048-33-4), ethylene glycol diacrylate (EGDA) (CAS number=2274-11-5) and sodium diallyloxyacetate (DAOAS) and more particularly from the group consisting of pentaerythritol tetraacrylate (PETA) and trimethylolpropane triacrylate (TMPTA).

The invention also relates to a process for preparing a linear or crosslinked copolymer as defined above, comprising the following successive steps:
[Chem 03] a step a) of providing:
   a solvent chosen from the group comprising water, ethanol, propanol, isopropanol, butanol, isobutanol, sec-butanol, tert-butanol, acetone, methyl ethyl ketone, cyclohexane, ethyl acetate or a mixture of two or more thereof,
   said monomer of formula (II) and said monomer of formula (I) in molar proportions such that the molar ratio R′A having as numerator the number of moles of compound of formula (I) and as denominator the number of moles of compound of formula (II) is greater than or equal to 0.4 and less than or equal to 3.0; and
   optionally said crosslinking monomer chosen from the group consisting of methylenebis(acrylamide), pentaerythritol tetraacrylate, trimethylolpropane triacrylate, triallylamine, pentaerythritol triallyl ether, 1,6-hexanediol diacrylate, ethylene glycol diacrylate and sodium diallyloxyacetate, in molar proportions such that the molar ratio R′B having as numerator the number of moles of crosslinking monomer and as denominator the sum of the numbers of moles of compound of formula (I) and of compound of formula (II), is greater than or equal to 0.001 and less than or equal to 0.007;
[Chem 04] a step b) of adding all of the moles of compound of formula (II) to the solvent provided in step a) to form a solution or dispersion of said compound of formula (II),
[Chem 05] optionally a step c) of partially or completely neutralizing the compound of formula (II), by adding a base to said solution or dispersion formed in step b), to form a solution or dispersion of said partially or completely neutralized compound of formula (II);
[Chem 06] a step d) of adding either part or all of the moles of compound of formula (I) provided in step a) to said solution or dispersion formed in step b) or to said solution or dispersion formed in step c), to form a solution or dispersion of said compound of formula (II), optionally partially or completely neutralized, and of said compound of formula (I);
[Chem 07] optionally a step e) of adding either part or all of the moles of crosslinking monomer to said solution or dispersion obtained in step d), to form a solution or dispersion of said compound of formula (II), optionally partially or completely neutralized, of said compound of formula (I) and optionally of said crosslinking monomer;
[Chem 08] a step f) of deoxygenating said solution or dispersion obtained in step d), or in optional step e) respectively, by sparging an inert gas, more particularly nitrogen, through it for a period of greater than or equal to 30 minutes and less than or equal to 90 minutes, to obtain a deoxygenated solution or dispersion of said compound of formula (II), optionally partially or completely neutralized, of said compound of formula (I) and optionally of said crosslinking monomer;
[Chem 09] a step g) of heating said solution or dispersion obtained in step f) at a temperature above or equal to 50° C. and below or equal to 95° C., to obtain a deoxygenated and heated solution or dispersion of said compound of formula (II), optionally partially or completely neutralized, of said compound of formula (I) and optionally of said crosslinking monomer;
[Chem 10] a step h) of polymerizing the monomers present in said deoxygenated and heated solution or dispersion obtained in step g), by adding a polymerization initiator, more particularly dilauroyl peroxide or 2,2′-azobis(2-methylpropionamidine) dihydrochloride and maintaining at a polymerization temperature above or equal to 50° C. and below or equal to 95° C., for a period greater than or equal to 150 minutes and less than or equal to 300 minutes, to obtain a precipitate of said expected, optionally crosslinked copolymer and optionally of said unreacted monomer of formula (II) in said solvent provided in step a);
[Chem 11] optionally a step i) of adding to said solvent obtained in step h), comprising the precipitate of said expected, optionally crosslinked copolymer and optionally said unreacted monomer of formula (II), the remainder of said compound of formula (I) and optionally the remainder of said crosslinking monomer provided in step a), and then polymerizing the remainder of the monomers present by adding a polymerization initiator, particularly dilauroyl peroxide or 2,2′-azobis(2-methylpropionamidine) dihydrochloride while maintaining the temperature at a temperature above or equal to 50° C. and below or equal to 95° C., for a period greater than or equal to 150 minutes and less than or equal to 300 minutes, to obtain a dispersion of said expected, optionally crosslinked copolymer;
[Chem 12] a step j) of cooling said dispersion of said optionally crosslinked copolymer obtained in step i) to a temperature above or equal to 15° C. and below or equal to 30° C.; and
[Chem 13] either a step k) of filtering said cooled dispersion obtained in step j) to collect said expected, optionally crosslinked copolymer, followed by a step 1) of drying said collected optionally crosslinked copolymer,
[Chem 14] or a step m) of spraying said cooled dispersion obtained in step j), in order to obtain said expected, optionally crosslinked copolymer in powder form.

According to a particular aspect of the process for preparing the linear or crosslinked copolymer as defined above, step b), optional step c), step d) and optional step e) are simultaneous and constitute a step B).

According to another particular aspect of the process for preparing the linear or crosslinked copolymer as defined above, the solvent provided in step a) is either water, or tert-butanol or a tert-butanol/water mixture.

According to this particular aspect, said process for preparing the linear or crosslinked copolymer more particularly comprises the following successive steps:
a step a) of providing:
   a tert-butanol/water mixture comprising, per 100% by weight, a weight proportion of greater than or equal to 90% and less than or equal to 98% by weight of tert-butanol and a weight proportion of greater than or equal to 2% by weight and less than or equal to 10% by weight of water;
   said monomer of formula (II) and said monomer of formula (I) in molar proportions such that the molar ratio R′A having as numerator the number of moles of compound of formula (I) and as denominator the number of moles of compound of formula (II) is greater than or equal to 1.0 and less than or equal to 3.0; and optionally D said crosslinking monomer chosen from the group consisting of pentaerythritol tetraacrylate and trimethylol propane triacrylate, in molar proportions such that the molar ratio R′B having as numerator the number of moles of crosslinking monomer and as denominator the sum of the numbers of moles of compound of formula (I) and of compound of formula (II), is greater than or equal to 0.003 and less than or equal to 0.005;
a step B) of adding, to said tert-butanol/water mixture provided in step a), all of the moles of said compound of formula (II), 50% of the moles of compound of formula (I) and optionally 50% of the moles of said crosslinking monomer to form a solution or a dispersion of said compound of formula (II), of said compound of formula (I) and optionally of said crosslinking monomer;
a step f) of deoxygenating said solution or dispersion obtained in step B), by sparging an inert gas through it for a period of greater than or equal to 45 minutes and less than or equal to 60 minutes, to obtain a deoxygenated solution or dispersion of said compound of formula (II), of said compound of formula (I) and optionally of said crosslinking monomer;
a step g) of heating said solution or dispersion obtained in step f) at a temperature above or equal to 50° C. and below or equal to 85° C., to obtain a deoxygenated and heated solution or dispersion of said compound of formula (II), of said compound of formula (I) and optionally of said crosslinking monomer;
a step h) of polymerizing the monomers present in said deoxygenated and heated solution or dispersion obtained in step g), by adding dilauroyl peroxide and maintaining at a polymerization temperature above or equal to 75° C. and below or equal to 85° C., for a period greater than or equal to 150 minutes and less than or equal to 200 minutes, to obtain a precipitate of said expected, optionally crosslinked copolymer and optionally of said unreacted monomer of formula (II) in said solvent provided in step a); and
a step i) of adding to said solvent obtained in step h), comprising the precipitate of said expected, optionally crosslinked copolymer and optionally said unreacted monomer of formula (II), the remainder of said compound of formula (I) and optionally the remainder of said crosslinking monomer provided in step a), and then polymerizing the remainder of the monomers present by adding dilauroyl peroxide and maintaining at a polymerization temperature above or equal to 75° C. and below or equal to 85° C., for a period greater than or equal to 150 minutes and less than or equal to 200 minutes, to obtain a dispersion of said expected, optionally crosslinked copolymer;
a step j) of cooling said dispersion of said optionally crosslinked copolymer obtained in step i) to a temperature above or equal to 15° C. and below or equal to 30° C.; and
either a step k) of filtering said cooled dispersion obtained in step j) to collect said expected, optionally crosslinked copolymer, followed by a step 1) of drying said collected optionally crosslinked copolymer,
or a step m) of spraying said cooled dispersion obtained in step j), in order to obtain said expected, optionally crosslinked copolymer in powder form.

The process as defined above makes it possible to obtain copolymers of natural origin which are advantageous replacements for the polymers of petroleum origin still used today in dermocosmetics or dermopharmacy.

This is why the invention also relates to the use of the linear or crosslinked copolymer as defined above, for thickening, stabilizing or emulsifying a cosmetic or pharmaceutical topical formulation or for suspending solid particles therein; and also to a cosmetic or pharmaceutical topical formulation, characterized in that it comprises, per 100.0% of its weight, from 0.1% to 10.0% by weight and more particularly from 0.5% to 5.0% by weight of the linear or crosslinked copolymer as defined above, as thickener, as stabilizer or as emulsifier of said cosmetic or pharmaceutical topical formulation or as agent capable of and intended for suspending solid particles within said cosmetic or pharmaceutical topical formulation.

The solid particles suspended in the cosmetic or pharmaceutical topical formulations which are the subject of the use or of the invention as defined above, have a regular or irregular geometry, and are generally in the form of pearls, beads, rods, flakes, lamellae or polyhedra. They are characterized by an average apparent diameter of between one micrometre and five millimetres, more particularly between ten micrometres and one millimetre. Examples of such solid particles include micas, iron oxide, titanium oxide, zinc oxide, aluminium oxide, talc, silica, kaolin, clays, boron nitride, calcium carbonate, magnesium carbonate, magnesium hydrogen carbonate, inorganic coloured pigments, polyamides, such as nylon-6, polyethylenes, polypropylenes, polystyrenes, polyesters, acrylic or methacrylic polymers, such as polymethyl methacrylates, polytetrafluoroethylene, crystalline or microcrystalline waxes, porous spheres, selenium sulfide, zinc pyrithione, starches, alginates, plant fibres, loofah particles and sponge particles.

In the cosmetic or pharmaceutical topical formulation which is the subject of the use or of the invention as defined above, the adjective topical means that said formulation is used by application to the skin, hair, scalp or mucous membranes, whether it is a direct application or an indirect application when the topical composition according to the invention is impregnated on a support intended to be brought into contact with the skin (paper, wipe, textile, transdermal device, etc.).

The cosmetic or pharmaceutical topical formulation which is the subject of the use or of the invention as defined above, may be in any physical form, for example in the form of an aqueous, aqueous-alcoholic or aqueous-glycolic gel; a solution; a powder; a suspension; or an emulsion, a microemulsion or a nanoemulsion, whether of water-in-oil, oil-in-water, water-in-oil-in-water or oil-in-water-in-oil type. They may be packaged in a bottle, in a pump-action bottle device, in pressurized form in an aerosol device, in a device equipped with an openwork wall such as a grill or in a device equipped with a ball applicator (known as a roll-on).

In general, the cosmetic or pharmaceutical topical formulation that is the subject of the use or of the invention as defined above may also comprise one or more pharmaceutically or cosmetically acceptable ingredient components, whether active principles or excipients, such as foaming, non-foaming or detergent surfactants, thickeners and/or gelling agents, stabilizers, solubilizers, film-forming compounds, solvents and cosolvents, hydrotropic agents, spring or mineral waters, plasticizers, emulsifiers and coemulsifiers, opacifiers, nacreous agents, superfatting agents, sequestrants, chelating agents, oils, waxes, antioxidants, fragrances, essential oils, preserving agents, conditioning agents, deodorants, whitening agents intended for bleaching body hairs and the skin, active principles intended to provide a treating and/or protective action with respect to the skin or the hair, sunscreens, mineral fillers or pigments, particles which provide a visual effect or which are intended for encapsulating active agents, exfoliating particles, texturing agents, optical brighteners or insect repellents.

The expression “cosmetically acceptable” used in the definition of abovementioned ingredients means, according to the European Economic Community Council Directive No. 76/768/EEC of Jul. 27, 1976, amended by Directive No. 93/35/EEC of Jun. 14, 1993, that said excipient (E) comprises water and any substance or preparation intended to be brought into contact with the various parts of the human body (epidermis, body hair and head hair system, nails, lips and genitals) or with the teeth and mucous membranes of the mouth, for the purpose, exclusively and mainly, of cleansing them, scenting them, modifying the appearance thereof and/or correcting body odours thereof and/or protecting them or keeping them in good condition.

The expression “pharmaceutically acceptable” used in the definition of the abovementioned ingredients means that these ingredients are listed in the Pharmacopoeia of the State in which the formulation is used.

Examples of foaming surfactants, optionally present in the cosmetic or pharmaceutical topical formulation which is the subject of the use or of the invention as defined above include anionic, cationic, amphoteric or nonionic foaming or detergent surfactants.

Foaming anionic surfactants that may be mentioned include alkali metal salts, alkaline-earth metal salts, ammonium salts, amine salts or amino alcohol salts:
of alkyl ether sulfates, such as the sodium C12-14 myristyl ether sulfate containing 3.5 mol of ethylene oxide, the sodium C12-14 lauryl ether sulfate containing 2 mol of ethylene oxide, the ammonium C12-14 lauryl ether sulfate containing 2 mol of ethylene oxide, the monoisopropylamine C12-14 lauryl ether sulfate containing 2 mol ethylene oxide in propylene glycol, the triisopropylamine C12-14 lauryl ether sulfate containing 2 mol of ethylene oxide in propylene glycol, the sodium C12-14 lauryl ether sulfate containing 3 mol of ethylene oxide, the ammonium C12-14 lauryl ether sulfate containing 3 mol of ethylene oxide, the sodium C12-15 lauryl ether sulfate containing 3 mol of ethylene oxide, the sodium C8-10 sodium lauryl ether sulfate containing 3 mol of ethylene oxide, the ammonium C8-10 lauryl ether sulfate containing 3 mol of ethylene oxide, the sodium C9-11 lauryl ether sulfate containing 2.5 mol of ethylene oxide, the ammonium C9-11 lauryl ether sulfate containing 2.5 mol of ethylene oxide, the ammonium C9-11 lauryl ether sulfate containing 2.5 mol of ethylene oxide in hexylene glycol;
of alkyl sulfates such as sodium lauryl sulfate, potassium lauryl sulfate, ammonium lauryl sulfate, magnesium lauryl sulfate, sodium cocoyl sulfate, potassium cocoyl sulfate, ammonium cocoyl sulfate, magnesium cocoyl sulfate;
alkylamido ether sulfates, alkylaryl polyether sulfates, monoglyceride sulfates, α-olefin sulfonates, paraffin sulfonates;
of alkyl phosphates, of alkyl ether phosphates, of alkyl sulfonates, of alkylamide sulfonates, of alkylaryl sulfonates, of alkyl carboxylates, of alkyl sulfosuccinates, of alkyl ether sulfosuccinates, of alkylamide sulfosuccinates, of alkyl sulfoacetates;
of N-acyl derivatives of amino acids, such as sodium lauroyl sarcosinate, potassium lauroyl sarcosinate, magnesium lauroyl sarcosinate, ammonium lauroyl sarcosinate, sodium lauroyl glycinate, potassium lauroyl glycinate, magnesium lauroyl glycinate, ammonium lauroyl glycinate, sodium cocoyl sarcosinate, potassium cocoyl sarcosinate, magnesium cocoyl sarcosinate, ammonium cocoyl sarcosinate, sodium cocoyl glycinate, potassium cocoyl glycinate, magnesium cocoyl glycinate, ammonium cocoyl glycinate, sodium cocoyl glutamate, potassium cocoyl glutamate, magnesium cocoyl glutamate, ammonium cocoyl glycinate, sodium cocoyl aspartate, potassium cocoyl aspartate, magnesium cocoyl aspartate, ammonium cocoyl aspartate; or mixtures comprising said N-acyl derivatives of amino acids for instance those sold under the brand name Proteol™OAT, Proteol™APL, Oramix™L30;
of acyl isethionates, such as sodium cocoyl isethionate, potassium cocoyl isethionate, magnesium cocoyl isethionate, ammonium cocoyl isethionate, sodium lauroyl isethionate, potassium lauroyl isethionate, magnesium lauroyl isethionate, ammonium lauroyl isethionate;
of N-acyl taurates, such as sodium methyl cocoyl taurate, potassium methyl cocoyl taurate, magnesium methyl cocoyl taurate, ammonium methyl cocoyl taurate or
of acyl lactylates.

Among the foaming amphoteric surfactants, mention may be made of.
alkyl betaines, such as lauryl betaine, cocoyl betaine, myristyl betaine;
alkylamido betaines, such as lauramidopropyl betaine, cocamidopropyl betaine sodium salt, 1-propanaminium, 3-amino-N-(carboxymethyl)-N,N-dimethyl-, or the composition sold under the brand name Amonyl™380 BA;
sultaines, alkylamidoalkyl sulfobetaines, or the composition sold under the brand name Amonyl™675 SB or
imidazolines derivatives, phosphobetaines, amphopolyacetates and amphopropionates.

Among the foaming cationic surfactants, mention may be made particularly of quaternary ammonium derivatives.

Among the foaming nonionic surfactants, mention may be made more particularly of alkyl polyglycosides including a linear or branched, saturated or unsaturated aliphatic radical, including from 8 to 16 carbon atoms, for instance n-octyl polyglucoside, n-decyl polyglucoside, n-undecylenyl polyglucoside, n-dodecyl polyglucoside, n-tetradecyl polyglucoside, n-hexadecyl polyglucoside, 1,12-dodecanediyl polyglucoside; ethoxylated hydrogenated castor oil derivatives, for instance the product sold under the INCI name PEG-40 hydrogenated castor oil; polysorbates, for instance Polysorbate 20, Polysorbate 40, Polysorbate 60, Polysorbate 70, Polysorbate 80 and Polysorbate 85; coconut kernel amides; N-alkylamines.

Examples of thickeners or gelling agents optionally present in the cosmetic or pharmaceutical topical formulation that is the subject of the use or of the invention as defined above, include:
optionally alkoxylated fatty esters of alkyl polyglycosides, such as ethoxylated esters of methyl polyglucoside for instance PEG-120 methyl glucose trioleate and PEG-120 methyl glucose dioleate sold, respectively, under the names Glucamate™ LT and Glucamate™ DOE120;
alkoxylated fatty esters such as PEG-150 pentaerythrityl tetrastearate sold under the name Crothix™ DS53,
PEG-55 propylene glycol oleate sold under the name ANTIL™ 141,
fatty-chain polyalkylene glycol carbamates such as PPG-14 laureth isophoryl dicarbamate sold under the name Elfacos™ T211, or PPG-14 palmeth-60 hexyl dicarbamate sold under the name Elfacos™ GT2125;
cellulose, cellulose derivatives such as methyl cellulose, ethyl cellulose, hydroxyethyl cellulose, hydroxypropyl cellulose, hydroxypropyl methylcellulose, carboxymethyl cellulose,
starch, hydrophilic starch derivatives,
polyurethanes,
silicates and phyllosilicates such as aluminium silicates, magnesium aluminium silicates or magnesium silicates, kaolinite, montmorillonite, illite, beidellite, saponite, bentonite, hectorite, vermiculite, serpentine, nacrite, amesite, nontronite, lizardite, sericite, halloysite, muscovite, paragonite, damourite, glauconite or celadonite;
branched or crosslinked, anionic, cationic or amphoteric polyelectrolytes such as the branched or crosslinked homopolymers of acrylic acid, methacrylic acid or 2-methyl-[(1-oxo-2-propenyl)amino]-1-propanesulfonic acid (AMPS), partially or completely salified with a sodium, ammonium or potassium salt; the copolymers of acrylic acid or methacrylic acid, partially or completely salified with a sodium, ammonium or potassium salt and of AMPS, partially or completely salified with a sodium, ammonium or potassium salt; the branched or crosslinked copolymers of acrylamide or vinylpyrolidone and of AMPS, partially or completely salified with a sodium, ammonium or potassium salt; the branched or crosslinked copolymers of AMPS, partially or completely salified with a sodium, ammonium or potassium salt and of (2-hydroxyethyl) acrylate, (2-hydroxyethyl) methacrylate, (2,3-dihydroxypropyl) acrylate or (2,3-dihydroxypropyl) methacrylate, hydroxyethylacrylamide, hydroxyethylmethacrylamide, N,N-dimethylacrylamide, N,N-dimethylmethacrylamide, N,N-diethylacrylamide, N,N-diethylmethacrylamide, N-methylacrylamide, N-ethylacrylamide, N-propylacrylamide, N-isopropylacrylamide, N-butylacrylamide, N-(tert-butyl)acrylamide, N-methylmethacrylamide, N-ethylmethacrylamide, N-propylmethacrylamide, N-isopropylmethacrylamide, N-butylmethacrylamide, N-(tert-butyl)methacrylamide, N,N-dipropylacrylamide or of tris(hydroxymethyl)acrylamidomethane (THAM); the branched or crosslinked copolymers of acrylic acid or methacrylic acid, partially or completely salified with a sodium, ammonium or potassium salt, and of (2-hydroxyethyl) acrylate, (2-hydroxyethyl) methacrylate, (2,3-dihydroxypropyl) acrylate, (2,3-dihydroxypropyl) methacrylate, hydroxyethylacrylamide, hydroxyethylmethacrylamide, N,N-dimethylacrylamide, N,N-dimethylmethacrylamide, N,N-diethylacrylamide, N,N-diethylmethacrylamide, N-methylacrylamide, N-ethylacrylamide, N-propylacrylamide, N-isopropylacrylamide, N-butylacrylamide, N-(tert-butyl)acrylamide, N-methylmethacrylamide, N-ethylmethacrylamide, N-propylmethacrylamide, N-isopropylmethacrylamide, N-butylmethacrylamide, N-(tert-butyl)methacrylamide, N,N-dipropylacrylamide or tris(hydroxymethyl)acrylamidomethane (THAM); the branched or crosslinked terpolymers of acrylic acid or methacrylic acid, partially or completely salified with a sodium, ammonium or potassium salt, of AMPS, partially or completely salified with a sodium, ammonium or potassium salt, and of (2-hydroxyethyl) acrylate, (2-hydroxyethyl) methacrylate, (2,3-dihydroxypropyl) acrylate, (2,3-dihydroxypropyl) methacrylate, hydroxyethylacrylamide, hydroxyethylmethacrylamide, N,N-dimethylacrylamide, N,N-dimethylmethacrylamide, N,N-diethylacrylamide, N,N-diethylmethacrylamide, N-methylacrylamide, N-ethylacrylamide, N-propylacrylamide, N-isopropylacrylamide, N-butylacrylamide, N-(tert-butyl)acrylamide, N-methylmethacrylamide, N-ethylmethacrylamide, N-propylmethacrylamide, N-isopropylmethacrylamide, N-butylmethacrylamide, N-(tert-butyl)methacrylamide, N,N-dipropylacrylamide, tris(hydroxymethyl)acrylamidomethane (THAM), acrylamide or methacrylamide; the branched or crosslinked copolymers of acrylic acid, methacrylic acid or AMPS, partially or completely salified with a sodium, ammonium or potassium salt, and of an alkyl acrylate or an alkyl methacrylate in which the carbon chain comprises between four and thirty carbon atoms and more particularly between ten and thirty carbon atoms; the branched or crosslinked terpolymers of AMPS, partially or completely salified with a sodium, ammonium or potassium salt, with at least one neutral monomer, and at least one monomer of formula CH₂═C(R′₃)—C(═O)—[CH₂—CH₂—O]ₙ—R′ in which R′₃ represents a hydrogen atom or a methyl radical, R′₄ represents a linear or branched, saturated or unsaturated alkyl radical containing from eight to thirty carbon atoms and more particularly a radical chosen from the elements of the group consisting of the following radicals: octyl, decyl, undecyl, dodecyl, tridecyl, tetradecyl, pentadecyl, hexadecyl, heptadecyl, octadecyl, nonadecyl, eicosyl, 2-ethylhexyl, 2-propylheptyl, 2-butyloctyl, 2-pentylnonyl, 2-hexyldecyl, 2-octyldodecyl, 4-methylpentyl, 5-methylhexyl, 6-methylheptyl, 15-methylpentadecyl, 16-methylheptadecyl, 2-hexyloctyl, 2-octyldecyl or 2-hexyldodecyl, and n represents a number greater than or equal to one and less than or equal to fifty; the homopolymers of N,N,N-trimethyl-3-[(2-methyl-1-oxo 2-propenyl)amino]propanaminium, N,N,N-trimethyl-3-[(1-oxo-2-propenyl)amino]propanaminium, diallyldimethylammonium, N,N,N-trimethyl-2-[(2-methyl-1-oxo-2-propenyl)]ethanaminium, and more particularly N,N,N-trimethyl-3-[(2-methyl-1-oxo-2-propenyl)amino]propanaminium chloride (MAMPTAC™) N,N,N-trimethyl-3-[(1-oxo-2-propenyl)amino]propanaminium chloride (APTAC™) diallyldimethylammonium chloride (DADMAC™), or N,N,N-trimethyl-2-[(2-methyl-1-oxo-2-propenyl)]ethanaminium (MADQUAT™); the branched or crosslinked copolymers of N,N,N-trimethyl-3-[(2-methyl-1-oxo-2-propenyl) amino]propanaminium chloride, N,N,N-trimethyl-3-[(1-oxo-2-propenyl)amino]propanaminium chloride, diallyldimethylammonium chloride or N,N,N-trimethyl-2-[(2-methyl-1-oxo-2-propenyl)]ethanaminium with acrylamide, methacrylamide, vinylpyrolidone, (2-hydroxyethyl) acrylate, (2-hydroxyethyl) methacrylate, (2,3-dihydroxypropyl) acrylate, (2,3-dihydroxypropyl) methacrylate, hydroxyethylacrylamide, N,N-dimethylacrylamide, N,N-dimethylmethacrylamide, N,N-diethylacrylamide, N,N-diethylmethacrylamide, N-methylacrylamide, N-ethylacrylamide, N-propylacrylamide, N-isopropylacrylamide, N-butylacrylamide, N-(tert-butyl)acrylamide, N-methylmethacrylamide, N-ethylmethacrylamide, N-propylmethacrylamide, N-isopropylmethacrylamide, N-butylmethacrylamide, N-(tert-butyl)methacrylamide, N,N-dipropylacrylamide or tris(hydroxymethyl)acrylamidomethane (THAM);
polymers sold under the brand names CARBOPOL™, PEMULEN™, ARISTOFLEX™, ARISTOFLEX™AVC, ARISTOFLEX™AVS, ARISTOFLEX™HMB, SEPIMAX™ Zen, SEPIMAX™ C, SEPIGEL™ 305, SEPIGEL™ 501, SEPIGEL™ 502, SIMULGEL™ 600, SIMULGEL™ EG, SIMULGEL™ EPG, SIMULGEL™ NS, SIMULGEL™ INS 100, SIMULGEL™ FL, SIMULGEL™ SMS 88, SIMULGEL™ 800, SIMULGEL™ A, SEPIPLUS™ 400, SEPIPLUS™ 250, SEPIPLUS™ S, SEPIPLUS™ NUDE, SEPILIFE™ G305, FlOCARE™ ET 25, FlOCARE™ ET 75, FlOCARE™ ET 26, FlOCARE™ ET 30, FlOCARE™ ET 58, FlOCARE™ PSD 30, VISCOLAM™ AT 64, VISCOLAM™ AT 100P, VISCOLAM™ AT EF, NOVEMER™ EC-1, NOVEMER™ EC-2, COSMEDIA™ SP, COSMEDIA™ ACE, SEPINOV™ EMT 10, SEPINOV™ WEO and SEPINOV™ P88;
thickeners or gelling agents of non-crosslinked polysaccharide type such as glucans or glucose homopolymers, glucomannoglucans, xyloglycans, galactomannans of which the degree of substitution (DS) of the D-galactose units on the main D-mannose chain is between 0 and 1, and more particularly between 1 and 0.25, such as galactomannans originating from cassia gum (DS=1/5), locust bean gum (DS=1/4), tara gum (DS=1/3), guar gum (DS=1/2) or fenugreek gum (DS=1); sulfated galactans and more particularly carrageenans and agar, uronans and more particularly algins, alginates and pectins, heteropolymers of monosaccharides and of uronic acids, and more particularly xanthan gum, gellan gum, gum arabic exudates and karaya gum exudates, glucosaminoglycans.

The solubilizers used in the context of the present invention are chemical substances or compositions which make it possible to solubilize hydrophobic substances that are insoluble in water, the aqueous phase or in the aqueous-alcoholic phase or in the aqueous-glycolic phase, such as fragrancing and flavouring substances. Examples of such agents optionally present in the cosmetic or pharmaceutical topical formulation that is the subject of the use or of the invention as defined above, include:
polysorbates, such as Polysorbate 20, Polysorbate 60 and Polysorbate 80;
alkyl polyglycoside compositions in which the linear or branched alkyl chain comprises from 4 to 10 carbon atoms, such as n-butyl polyglucoside, n-butyl polyxyloside, n-pentyl polyglucoside, n-pentyl polyxyloside, n-hexyl polyglucoside, n-hexyl polyxyloside, n-heptyl polyglucoside, n-heptyl polyxyloside, n-octyl polyglucoside, n-octyl polyxyloside, n-nonyl polyglucoside, n-nonyl polyxyloside, n-decyl polyglucoside, n-decyl polyxyloside;
ethoxylated fatty alcohols of formula R10-(OE)_{n′}-H, where R10 represents a linear or branched, saturated or unsaturated aliphatic radical containing from 12 to 22 carbon atoms, preferably from 12 to 18 carbon atoms, and more particularly from 12 to 16 carbon atoms, and n′ represents an integer greater than or equal to 5 and less than or equal to 200, more particularly greater than or equal to 5 and less than or equal to 100, more particularly greater than or equal to 10 and less than or equal to 100; for example compounds of formula R10-(OE)_{n′}-H, where R10 represents the dodecyl radical and n′ represents an integer greater than or equal to 7 and less than or equal to 25;
polyethoxylated fatty acids of formula R20-C(═O)—(OE)_{m′}, where R20 represents a linear or branched, saturated or unsaturated aliphatic radical containing 12 to 22 carbon atoms, more particularly 12 to 18 and m′ represents an integer greater than or equal to 10 and less than or equal to 100, more particularly greater than or equal to 15 and less than or equal to 100, and even more particularly greater than or equal to 15 and less than or equal to 50;
hydrogenated and ethoxylated oils, and more particularly those comprising at least one triglyceride or one diglyceride or one monoglyceride such as hydrogenated and ethoxylated castor oil containing 40 mol of ethylene oxide sold under the name PEG-40 hydrogenated castor oil;
As examples of emulsifying agents optionally present in the cosmetic or pharmaceutical topical formulation which is the subject of the use or of the invention as defined above, mention may be made of those selected from the elements of the group consisting of alkyl polyglycoside compositions and in particular alkyl polyglucosides and alkyl polyxylosides, alkyl polyglycoside and fatty alcohol compositions, polyglycerol esters and in particular decaglycerol oleate, decaglycerol isostearate, decaglycerol monolaurate, decaglycerol monolinoleate, decaglycerol monomyristate, alkoxylated polyglycerol esters, polyglycol polyhydroxystearates, polyglycerol polyhydroxystearates, alkoxylated polyglycerol polyhydroxystearates.

Examples of oils optionally present in the cosmetic or pharmaceutical topical formulation that is the subject of the use or of the invention as defined above, include:
linear alkanes containing from 11 to 19 carbon atoms, such as undecane, dodecane, tridecane, tetradecane, pentadecane, hexadecane, heptadecane, octadecane and nonadecane;
branched alkanes containing from 7 to 40 carbon atoms, such as isododecane, isopentadecane, isohexadecane, isoheptadecane, isooctadecane, isononadecane or isoeicosane, or mixtures of some thereof such as those mentioned below and identified by their INCI name: C₇₋₈ isoparaffin, C₈₋₉ isoparaffin, C₉₋₁₁ isoparaffin, C₉₋₁₂ isoparaffin, C₉₋₁₃ isoparaffin, C₉₋₁₄ isoparaffin, C₉₋₁₆ isoparaffin, C₁₀₋₁₁ isoparaffin, C₁₀₋₁₂ isoparaffin, C₁₀₋₁₃ isoparaffin, C₁₁₋₁₂ isoparaffin, C₁₁₋₁₃ isoparaffin, C₁₁₋₁₄ isoparaffin, C₁₂₋₁₄ isoparaffin, C₁₂₋₂₀ isoparaffin, C₁₃₋₁₄ isoparaffin, C₁₃₋₁₆ isoparaffin;
cycloalkanes optionally substituted with one or more linear or branched alkyl radicals;
white mineral oils, such as those sold under the following names: MARCOL™ 52, MARCOL™ 82, DRAKEOL™ 6VR, EOLANE™ 130 and EOLANE™ 150;
hemisqualane (or 2,6,10-trimethyldodecane; CAS number: 3891-98-3), squalane (or 2,6,10,15,19,23-hexamethyltetracosane), hydrogenated polyisobutene or hydrogenated polydecene;
mixtures of alkanes comprising from 15 to 19 carbon atoms, said alkanes being linear alkanes, branched alkanes and cycloalkanes, and more particularly the mixture (Mi) which comprises, per 100% of its weight, a weight proportion of branched alkanes of greater than or equal to 90% and less than or equal to 100%; a weight proportion of linear alkanes of greater than or equal to 0% and less than or equal to 9%, and more particularly less than 5%, and a weight proportion of cycloalkanes of greater than or equal to 0% and less than or equal to 1%, for example the mixtures sold under the name EMOGREEN™ L15 or EMOGREEN™ L19;
the fatty alcohol ethers of formula Z1-O—Z2, in which Z1 and Z2, which may be identical or different, represent a linear or branched alkyl radical containing from 5 to 18 carbon atoms, for example dioctyl ether, didecyl ether, didodecyl ether, dodecyl octyl ether, dihexadecyl ether, (1,3-dimethylbutyl) tetradecyl ether, (1,3-dimethylbutyl) hexadecyl ether, bis(1,3-dimethylbutyl) ether or dihexyl ether;
monoesters of fatty acids and of alcohols of formula R′1(C═O)—O—R′2 in which R′1(C═O)— represents a linear or branched, saturated or unsaturated acyl radical containing from 8 to 24 carbon atoms and R′2 represents, independently of R′1, a linear or branched, saturated or unsaturated hydrocarbon chain containing from 1 to 24 carbon atoms, for example methyl laurate, ethyl laurate, propyl laurate, isopropyl laurate, butyl laurate, 2-butyl laurate, hexyl laurate, methyl cocoate, ethyl cocoate, propyl cocoate, isopropyl cocoate, butyl cocoate, 2-butyl cocoate, hexyl cocoate, methyl myristate, ethyl myristate, propyl myristate, isopropyl myristate, butyl myristate, 2-butyl myristate, hexyl myristate, octyl myristate, methyl palmitate, ethyl palmitate, propyl palmitate, isopropyl palmitate, butyl palmitate, 2-butyl palmitate, hexyl palmitate, octyl palmitate, methyl oleate, ethyl oleate, propyl oleate, isopropyl oleate, butyl oleate, 2-butyl oleate, hexyl oleate, octyl oleate, methyl stearate, ethyl stearate, propyl stearate, isopropyl stearate, butyl stearate, 2-butyl stearate, hexyl stearate, octyl stearate, methyl isostearate, ethyl isostearate, propyl isostearate, isopropyl isostearate, butyl isostearate, 2-butyl isostearate, hexyl isostearate, or isostearyl isostearate;
diesters of fatty acids and of glycerol of formula R′3-(C═O)—O—CH₂—CH(OH)—CH₂—O—(C═O)—R′4 and of formula R′5-(C═O)—O—CH₂—CH(O—(C═O)—R′6)-CH₂—OH in which R′3-(C═O), R′4-(C═O), R′5-(C═O) and R′6-(C═O), which may be identical or different, represent a linear or branched, saturated or unsaturated acyl group containing from 8 to 24 carbon atoms;
triesters of fatty acids and of glycerol of formula R′7-(C═O)—O—CH₂—CH(O—(C═O)—R′8)-CH₂—O—(C═O)—R′9, in which R′7-(C═O), R′8-(C═O) and R′9-(C═O), which may be identical or different, represent a linear or branched, saturated or unsaturated acyl group containing from 8 to 24 carbon atoms;
plant oils, such as phytosqualane or sweet almond oil, coconut oil, castor oil, jojoba oil, olive oil, rapeseed oil, groundnut oil, sunflower oil, wheat germ oil, corn germ oil, soybean oil, cottonseed oil, alfalfa oil, poppy seed oil, pumpkin seed oil, evening primrose oil, millet oil, barley oil, rye oil, safflower oil, candlenut oil, passionflower oil, hazelnut oil, palm oil, apricot kernel oil, calophyllum oil, sisymbrium oil, avocado oil, calendula oil or oils resulting from flowers or vegetables;
ethoxylated plant oils.

In the present invention, the term “wax” denotes a water-insoluble compound or mixture of compounds, which has a solid appearance at a temperature greater than or equal to 45° C. As examples of waxes that may be present in the cosmetic or pharmaceutical topical formulation that is a subject of the use or of the invention as defined above, mention may be made of beeswax, carnauba wax, candelilla wax, ouricury wax, Japan wax, cork fibre wax, sugarcane wax, paraffin wax, lignite wax, lanolin wax, ozokerite wax, polyethylene wax or silicone wax; plant waxes or microcrystalline waxes, fatty alcohols, fatty acids and glycerides that are solid at room temperature, such as shea butter or cocoa butter.

As examples of natural tspring or mineral waters that may be present in the topical cosmetic or pharmaceutical topical formulation which is the subject of the use or of the invention as defined above, mention may be made of Avène water, Vittel water, Uriage water, La Roche Posay water, La Bourboule water, Enghien-les-bains water, Saint-Gervais-les-bains water, Néris-les-bains water, Allevard-les-bains water, Digne water, Maizieres water, Neyrac-les-bains water, Lons le Saunier water, Rochefort water, Saint Christau water, Fumades water, Tercis-les-Bains water, Bagnères-de-Bigorres water, Eugénie-les-Bains water, Challes-les-Eaux water, Volvic water, Vals water, Vernière water, Aix les Bains water, Alet water, Abatilles water, Arcens water, Arvie water, Asperjoc water, Badoit water, Cilaos water, Contrexéville water, Evian water, Hépar water, Jouvence water, Mont-Roucous water, Ogeu water, Orezza water, Parot water, Perrier water, Plantcoët water, Quézac water, Rozana water, Saint-Alban-Les-Eaux water, Saint-Amand-les Eaux water, Saint-Georges water, Saint-Géron water, Sainte-Marguerite water, Saint-Yorre water, Salvetat water, Teissières-lès-Bouliès water, Thonon water, Treignac water, Courmayeur water, San Benedetto water, San Pellegrino water or Vichy basin water.

As examples of deodorant agents that may be present in the topical cosmetic or pharmaceutical formulation which is the subject of the use or of the invention as defined above, mention may be made of alkali metal silicates, zinc salts such as zinc sulfate, zinc gluconate, zinc chloride or zinc lactate; quaternary ammonium salts such as cetyltrimethylammonium salts or cetylpyridinium salts; glycerol derivatives such as glyceryl caprate, glyceryl caprylate and polyglyceryl caprate; capryloyl glycine; 1,2-decanediol, 1,3-propanediol; salicylic acid; sodium bicarbonate; cyclodextrins; metallic zeolites; Triclosan™; aluminium salts such as aluminium bromohydrates, aluminium chlorohydrates, aluminium chloride, aluminium sulfate or aluminium lactate; mixed aluminium zirconium chlorohydrates such as aluminium zirconium chlorohydrates, aluminium zirconium trichlorohydrate, aluminium zirconium tetrachlorohydrate, aluminium zirconium pentachlorohydrate or aluminium zirconium octachlorohydrate; sodium aluminium lactate; complexes of aluminium chlorohydrates and of diols such as the aluminium chlorohydrate-glycol or aluminium chlorohydrate-propylene glycol complexes, the aluminium dichlorohydrate-propylene glycol complex, the aluminium sesquichlorohydrate-propylene glycol complex, the aluminium chlorohydrate-polyethylene glycol complex, the aluminium dichlorohydrate-polyethylene glycol complex, or the aluminium sesquichlorohydrate-polyethylene glycol complex.

As hydrotropic agents that may be present in the cosmetic or pharmaceutical topical formulation which is the subject of the use or of the invention as defined above, mention may be made of xylene sulfonates, cumenes sulfonates, hexylpolyglucoside, 2-ethylhexylpolyglucoside or n-heptylpolyglucoside.

As antioxidants that may be present in the cosmetic or pharmaceutical topical formulation which is the subject of the use or of the invention as defined above, mention may be made of EDTA (ethylenediamine tetraacetic acid) and salts thereof, citric acid, tartaric acid, oxalic acid, BHA (butylhydroxyanisole), BHT (butylhydroxytoluene), tocopherol derivatives such as tocopherol acetate, DISSOLVINE™ GL 47S or CONTACTICEL™.

As examples of sunscreens that may be present in the cosmetic or pharmaceutical topical formulation which is the subject of the use or of the invention as defined above, mention may be made of all those listed in the amended cosmetics directive 76/768/EEC, Annex VII.

As examples of solubilizing agents for organic screening agents that may be present in the cosmetic or pharmaceutical topical formulation which is the subject of the use or of the invention as defined above, mention may be made of LANOL™ 37T, DUB™ 810PGM, DUB™ DIS, DUB™DIPA, DUB™ DNPG or DUB™ SYNERSOL.

As an example of a mineral sunscreen that may be present in the cosmetic or pharmaceutical topical formulation which is the subject of the use or of the invention as defined above, mention may be made of titanium oxides, zinc oxides, cerium oxides, zirconium oxides, yellow, red or black iron oxides, or chromium oxides.

As examples of solvents and cosolvents that may be present in the cosmetic or pharmaceutical topical formulation which is the subject of the use or of the invention as defined above, mention may be made of glycerol, diglycerol, glycerol oligomers, ethylene glycol, propylene glycol, butylene glycol, hexylene glycol, diethylene glycol, xylitol, erythritol, sorbitol, water-soluble alcohols such as ethanol, isopropanol or butanol, mixtures of water and of said organic solvents, propylene carbonate, ethyl acetate, benzyl alcohol and dimethyl sulfoxide (DMSO).

As examples of agents for improving the skin penetration that may be present in the cosmetic or pharmaceutical topical formulation which is the subject of the use or of the invention as defined above, mention may be made of glycol ethers, for instance ethylene glycol monomethyl ether, ethylene glycol monoethyl ether, ethylene glycol monopropyl ether, ethylene glycol monoisopropyl ether, ethylene glycol monobutyl ether, ethylene glycol monophenyl ether, ethylene glycol monobenzyl ether, diethylene glycol monomethyl ether, diethylene glycol monoethyl ether and diethylene glycol mono(n-butyl) ether, diethylene glycol monoethyl ether (or Transcutol-P), fatty acids such as oleic acid, fatty acid esters of glycerol, for instance glyceryl behenate, glyceryl palmitostearate, behenoyl macroglycerides, polyoxyethylene (2) stearyl ether, polyoxyethylene (2) oleyl ether, terpenes, for instance D-limonene, and essential oils, for instance the essential oil of eucalyptus.

As examples of stabilizers that may be present in the cosmetic or pharmaceutical topical formulation which is the subject of the use or of the invention as defined above, mention may be made of microcrystalline waxes, and more particularly ozokerite, mineral salts such as sodium chloride or magnesium chloride, silicone polymers such as polysiloxane polyalkyl polyether copolymers.

As examples of active principles that may be present in the cosmetic or pharmaceutical topical formulation which is the subject of the use or of the invention as defined above, mention may be made of:
vitamins and derivatives thereof, notably esters thereof, such as retinol (vitamin A) and esters thereof (for example retinyl palmitate), ascorbic acid (vitamin C) and esters thereof, sugar derivatives of ascorbic acid (for example ascorbyl glucoside), tocopherol (vitamin E) and esters thereof (for instance tocopheryl acetate), vitamins B3 or B10 (niacinamide and derivatives thereof);
compounds with lightening or depigmenting action on the skin, such as ω-undecelynoyl phenylalanine sold under the name Sepiwhite™ MSH, Sepicalm™ VG, the glyceryl monoester and/or diester of ω-undecelynoyl phenylalanine, ω-undecelynoyl dipeptides, arbutin, kojic acid, hydroquinone;
compounds with a calmative action, such as Sepicalm™ S, allantoin and bisabolol;
analgesic or antiinflammatory agents such as acetaminophen, aspirin, salicylic acid, methyl salicylate, choline salicylate, glycol salicylate, 1-menthol, camphor, mefenamic acid, flufenamic acid, indomethacin, protizinic acid, fentiazac, tolmetin, phenylbutazone, oxyphenbutazone, clofezone, pentazocine, mepirizole, hydrocortisone, cortisone, dexamethasone, fluocinolone, triamcinolone, medrysone, prednisolone, flurandrenolide, prednisone, halcinonide, methylprednisolone, fludrocortisone, corticosterone, paramethasone and betamethasone; (hetero)aryl acetic acid or 2-(hetero)aryl propionic acid such as diclofenac, tiaprofenic acid, alminoprofen, etodolac, flurbiprofen, ibuprofen, ketoprofen or naproxen.
antiseptic agents such as cetrimide, povidone-iodine, chlorhexidine, viodine, benzalkonium chloride, benzoic acid, nitrofurazone, benzoyl peroxide, hydrogen peroxide, hexachlorophene, phenol, resorcinol or cetylpyridinium chloride;
insecticidal agents such as trichlorfon, triflumuron, fenthion, bendiocarb, cyromazine, diflubenzuron, dicyclanil, fluazuron, amitraz, deltamethrin, cypermethrin, chlorfenvinphos, flumethrin, ivermectin, abamectin, avermectin, doramectin, moxidectin, zeta-cypermethrin, diazinon, spinosad, imidacloprid, nitenpyram, pyriproxyfen, fipronil, cythioate, lufenuron, selamectin, milbemycin oxime, chlorpyrifos, coumaphos, propetamphos, alpha-cypermethrin, high cis cypermethrin, ivermectin, diflubenzuron, cyclodiene, carbamate or benzoylurea.
antimicrobial agents such as sulfonamides, neomycin, tobramycin, gentamycin, amikacin, kanamycin, spectinomycin, paromomycin, netilmicin, polypeptides, cephalosporins or oxazolidinones such as ciprofloxacin, levofloxacin or ofloxacin;
compounds with a moisturizing action such as urea, hydroxyureas, glycerol, polyglycerols, glyceryl glucoside, diglyceryl glucoside and polyglyceryl glucosides or xylityl glucoside;
polyphenol-rich plant extracts, for instance grape extracts, pine extracts, wine extracts and olive extracts;
compounds with slimming or lipolytic action, such as caffeine or derivatives thereof, Adiposlim™, Adipoless™, and fucoxanthin;
N-acyl proteins, N-acyl peptides, such as Matrixil™, N-acyl amino acids, partial hydrolysates of N-acyl proteins, amino acids, peptides, complete protein hydrolysates,
plant extracts, for instance soybean extracts, for example Raffermine™, wheat extracts, for instance Tensine™ or Gliadine™, plant extracts, such as tannin-rich plant extracts and isoflavone-rich plant extracts or terpene-rich plant extracts; freshwater or saltwater algal extracts, marine plant extracts; marine extracts in general such as corals;
essential waxes;
bacterial extracts;
ceramides;
phospholipids;
compounds with antimicrobial action or with purifying action, such as Lipacide™ C8G, Lipacide™ UG, Sepicontrol™ A5; Octopirox™ or Sensiva™ SC50;
compounds with an energizing or stimulating property such as Physiogenyl™
panthenol and derivatives thereof such as Sepicap™ MP;
anti-ageing active agents such as Sepilift™ DPHP, Lipacide™ PVB, Sepivinol™, Sepivital™, Manoliva™, Phyto-Age™, Timecode™ or Survicode™;
anti-photoageing active agents; agents for protecting the integrity of the dermoepidermal junction; agents for increasing the synthesis of components of the extracellular matrix, such as collagen, elastins and glycosaminoglycans;
active agents acting favourably on chemical cellular communication, such as cytokines, or on physical cellular communication, such as integrins;
active agents which create a “heating” sensation on the skin, such as skin microcirculation activators (such as nicotinic acid derivatives) or products which create a “freshness” sensation on the skin (such as menthol and derivatives thereof);
active agents which improve the skin microcirculation, for example venotonic agents;
draining active agents; decongesting active agents, such as extracts of Ginkgo biloba, ivy, common horse chestnut, bamboo, ruscus, butcher's-broom, Centella asiatica, fucus, rosemary or willow;
agents intended for treating head hair or body hair, for example agents for protecting the melanocytes of the hair follicle, mimetics of DOPAchrome tautomerase activity, synthetic SOD mimetic molecules, for example manganese complexes, antioxidant compounds, for example cyclodextrin derivatives, siliceous compounds derived from ascorbic acid, lysine pyrrolidone carboxylate or arginine pyrrolidone carboxylate, combinations of monoesters and diesters of cinnamic acid and of vitamin C;
agents for bronzing or tanning the skin, for instance dihydroxyacetone (DHA), erythrulose, meso-tartaric aldehyde, glutaraldehyde, glyceraldehyde, alloxan, ninhydrin, plant extracts, for instance extracts of redwoods of the genus Pterocarpus and of the genus Baphia, for instance Pterocarpus santalinus, Pterocarpus osun, Pterocarpus soyauxii, Pterocarpus erinaceus, Pterocarpus indicus or Baphia nitida;
agents known for their action in facilitating and/or accelerating the bronzing or tanning of human skin, or for their action in colouring human skin, for example carotenoids (and more particularly β-carotene and γ-carotene), the products sold under the brand name “Carrot oil” (INCI name: to color human skin, e.g. caratenoids (in particular beta-carotene and gamma-carotene), products sold under the brand names Carrot oil, SunTan Accelerator™, Zymo Tan Complex, MelanoBronze™, Monk's pepper extract, Unipertan VEG-24/242/2002, Try-Excell™, Actibronze™, Tyrostan™, Tyrosinol, InstaBronze™, Tyrosilane, Exymol, Bronzing SF Peptide powder, Melitane, Melatimes Solutions™, Tanositol™, Thalitan™, Phycosaccharide™ AG, Melactiva™ and Biotanning™
As fragrancing or flavouring substances that may be present in the topical cosmetic or pharmaceutical topical formulation which is the subject of the use or of the invention as defined above, mention may be made of those extracted from flowers such as rose, jasmine, tuberose, champak, mimosa, carnation, osmanthus, narcissus, lavender or gardenia or frangipani flowers, ylang-ylang flowers, lotus flowers, acacia flowers, sweet orange blossom, bitter orange blossom or neroli blossom; those extracted from leaves, moss, bark, resin or buds such as blackcurrant buds; oakmoss, beech moss or lichen; acacia leaves, basil leaves, valerian leaves, gentian leaves, violet leaves, geranium leaves, labdanum leaves, rosemary leaves, patchouli leaves or verbena leaves; cinnamon bark, ash bark, cassia bark, cascarilla bark; sandalwood, cedarwood, rosewood, agarwood, birch wood, guaiac wood; Peru balsam, Tolu balsam, benzoin resin, myrrh, labdanum resin, elemi resin, olibanum, opoponax, guggul, those extracted from needles and branches of pine or spruce or fir; those extracted from tarragon, lemongrass, sage or thyme; those extracted from pods, beans or berries such as tonka bean, vanilla pods, cardamom, coriander, star anise, bitter almond, cumin, cloves, juniper berries; or those extracted from citrus fruits such as lemon, orange including limetta and bergamot, mandarin; those extracted from roots such as the roots of angelica, celery, cardamom, iris, sweet-flag, cactus, or vetiver.

As fragrancing or flavouring substances that may be present in the topical cosmetic or pharmaceutical topical formulation which is the subject of the use or of the invention as defined above, mention may also be made of those from yarrow, Acorus calamus, garlic, ajowan, amyris, dill, anise, angelica, tea tree, basil, Bay Saint Thomas, benzoin, bergamot, guaiac wood, ho wood, rosewood, sandalwood, siam wood, black birch, chamomile, camphor tree, cinnamon, cardamom, carrot, caraway, cedar, celery, sea fennel, cistus, lemon, citronella, clementine, kaffir lime oil, copaiba, coriander, cryptomeria, cumin, turmeric, cypress, frankincense, spruce oil, tarragon, fennel, fragonia, galbanum, wintergreen, juniper, geranium, ginger, clove or clove leaf, helichrysum, hyssop, iary, inula, katrafay, khella, kunzea, lavender, lavandin, mandarin, niaouli, peppermint, orange, grapefruit, rosemary, thyme, ylang ylang, ravintsara, sage, cabreuva, lemongrass, palmarosa, St. John's wort, jasmine, chamomile, melissa, pine, ginger, parsley, artemisia, hemp, hop, or else wild thyme.

As fragrancing or flavouring substances that may be present in the cosmetic or pharmaceutical topical formulation which is the subject of the use or of the invention as defined above, mention may also be made of musk, castoreum, civet, ambergris, beeswax absolute or hyraceum.

As chemical compounds that may be present in the cosmetic or pharmaceutical topical formulation which is the subject of the use or of the invention as defined above, mention may be made of:
terpene hydrocarbons (monoterpenes and sesquiterpenes) such as α-myrcene or β-myrcene, limonene, α-pinene or β-pinene, camphene, cadinene, cedrene, farnesene, caryophyllene, chamazulene, 1,1-dimethoxy-2,2,5-trimethyl-4-hexene, curcumene, crithmene, himachalenes, limonene, para-cymene, rose oxide or tetranorlabdane oxide; terpinenes, terpinolenes or vetivenes;
esters such as benzyl acetate, bornyl acetate, citronellyl acetate, cedryl acetate, dihydromyrcenyl acetate, dimethylbenzylcarbinyl acetate, ethyl acetate, farnesyl acetate, fenchyl acetate, hexyl acetate, geranyl acetate, isobutyl acetate, isononyl acetate, isopentyl acetate, isobornyl acetate, isopulegyl acetate, linalyl acetate, menthyl acetate, methyl phenyl carbinyl acetate, neryl acetate, nonyl acetate, 2-tert-butylcyclohexyl acetate, phenylethyl acetate, 4-tert-butylcyclohexyl acetate, prenyl acetate, styrallyl acetate, terpenyl acetate or vetiveryl acetate; methyl anthranilate, benzyl benzoate, isobutyl benzoate or linalyl benzoate, coumarin, ethyl butanoate, benzyl butanoate or isoamyl butanoate, benzyl butyrate, ethyl butyrate, isoamyl butyrate or linalyl butyrate; butyl cinnamate, allyl cinnamate or ethyl cinnamate; benzyl formate, citronellyl formate, hedione formate, geranyl formate or methyl formate; ethyl methylphenylglycinate, allyl amyl glycolate, allyl heptanoate, phenoxyethyl isobutyrate, cis-3-hexenyl isobutyrate, isoamyl methacrylate, ethyl naphtholate, hexyl neopentanoate, amyl propionate, alkylcyclohexyl propionate, allylcyclohexane propionate, linalyl propionate, styrallyl propionate or citronellyl propionate; methyl salicylate, benzyl salicylate or ethyl salicylate or hexyl tiglate;
alcohols and phenols such as benzyl alcohol, α-terpineol, anethole, carotol, chavicol, estragole, cineole, cinnamyl alcohol, citronellol, p-cresol, cumyl alcohol, 3,7-dimethyl-1-octanol, dimethyl benzyl carbinol, fenchyl alcohol, eucalyptol, farnesol, eugenol, isononyl alcohol, isoeugenol, guaiacol, geraniol, globulol, linalool, menthol, dihydromyrcenol, nerolidol, nerol, phenylethyl alcohol, safrole, isosafrole, phytol, isophytol, terpineol, tetrahydrolinalool, tetrahydromyrcenol, thymol, vetiverol, undecavertol;
aldehydes such as phenylacetaldehyde, salicylaldehyde, anisaldehyde, caprylaldehyde, cinnamaldehyde, hexylcinnamaldehyde; bourgeonal, citral, citronellal, hydroxycitronellal, citronellyloxyacetaldehyde, cyclamen aldehyde, cuminaldehyde, cyclal, 2,4-dimethyl-3-cyclohexene-1-carboxaldehyde, dodecanal, ethanal, octanal, decanal, geranials, helional, lactones such as γ-undecalactones, lilial, methyl-n-nonyl acetaldehyde, methyloctyl acetaldehyde, undecanal or vanillin;
ketones such as benzylacetone, 7-methyl-2H-benzo-1,5-dioxepin-3(4H)-one, carvone, camphor, civetone, damascones, damascenones, ethyl amyl ketone, ethyl hexyl ketone, geranylacetone, jasmone, irones, 3-hydroxy-2-methyl-4H-pyran-4-one, ethyl maltol, menthone, isomenthone, muscone, methylheptenone, ionones such as methyl ionone, 4-methylacetophenone, methyl pentyl ketone, methyl heptyl ketone, methyl hexyl ketone, α-isomethyl ionone, or methyl cedryl ketone;
ethers such as anethole, benzyl ethyl ether, cedryl methyl ether, p-cresyl methyl ether;
artificial musks derived from various nitro compounds, musk ambrettes, musk ketones, musk xylenes, macrocyclic musks,
nitriles such as trimethyl-3,5,7-octane(ene) nitriles and the α-substituted derivatives thereof, citronellyl nitrile, citronitrile, geranyl nitrile.

### EXAMPLES

The examples that follow illustrate the invention without, however, modifying it.

Preparation of a Tulipalin A-Itaconic Acid Copolymer (Molar Ratio RA=2), Crosslinked with Pentaerythritol Tetraacrylate (Molar Ratio RB=0.0040) According to the Invention (P1).

30.0 g of tert-butanol and 1.5 g of demineralized water are poured into a reactor with mechanical stirring. 6.50 g (0.05 mol) of itaconic acid, 4.91 g (0.05 mol) of Tulipalin A and 0.11 g (0.0003 mol) of pentaerythritol tetraacrylate are then added thereto. The dispersion obtained is deoxygenated by nitrogen sparging for fifty minutes and then heated to 80° C. 0.20 g of dilauroyl peroxide is then added thereto and the whole medium is maintained at 80° C. with mechanical stirring for three hours. 4.91 g (0.05 mol) of Tulipalin A are then added (to achieve the molar ratio RA=2) followed by 0.11 g (0.0003 mol) of pentaerythritol tetraacrylate (to achieve the molar ratio RB=0.0040) and 0.20 g of dilauroyl peroxide. The medium obtained is maintained at 80° C. with mechanical stirring for another three hours. It is then cooled to 35° C. and filtered on paper and the precipitate collected is finally dried in an oven at 50° C. under a vacuum of 20 mbar. 13.78 g of the expected copolymer (P1) are thus isolated, which corresponds to a final yield of 83.4%.

Preparation of a Tulipalin A-Itaconic Acid Copolymer (RA=3), Crosslinked with Pentaerythritol Tetraacrylate (RB=0.0039) According to the Invention (P2)

30.0 g of tert-butanol and 1.5 g of demineralized water are poured into a reactor with mechanical stirring. 4.88 g (0.0375 mol) of itaconic acid, 5.30 g (0.054 mol) of Tulipalin A and 0.10 g (0.0002838 mol) of pentaerythritol tetraacrylate are then added thereto. The dispersion obtained is deoxygenated by nitrogen sparging for 45 minutes and then heated to 80° C. 0.18 g of dilauroyl peroxide is then added thereto and the whole medium is maintained at 80° C. with mechanical stirring for three hours. 5.30 g (0.054 mol) of Tulipalin A are then added (to achieve the molar ratio RA=3), followed by 0.10 g (0.0002838 mol) of pentaerythritol tetraacrylate (to achieve the molar ratio RB=0.0039) and 0.18 g of dilauroyl peroxide are again added to the heterogeneous reaction medium. The medium obtained is maintained at 80° C. with mechanical stirring for another three hours. It is then cooled to 35° C. and filtered on paper and the precipitate collected is finally dried in an oven at 50° C. under a vacuum of 20 mbar. 12.29 g of the copolymer (P2) are thus isolated, which corresponds to a final yield of 78.4%.

Preparation of a Tulipalin A-Itaconic Acid Copolymer (RA=0.4), Crosslinked with Pentaerythritol Tetraacrylate (RB=0.0069) According to the Invention (P3)

30.0 g of tert-butanol and 1.5 g of demineralized water are poured into a reactor with mechanical stirring. 9.30 g (0.0715 mol) of itaconic acid, 1.40 g (0.001425 mol) of Tulipalin A and 0.09 g (0.0002572 mol) of pentaerythritol tetraacrylate are then added thereto. The dispersion obtained is deoxygenated by nitrogen sparging for 60 minutes and then heated to 80° C. 0.17 g of dilauroyl peroxide is then added thereto and the whole medium is maintained at 80° C. with mechanical stirring for three hours. 1.40 g (0.01425 mol) of Tulipalin A are then added (to achieve the molar ratio RA=0.4), followed by 0.09 g (0.0002572 mol) of pentaerythritol tetraacrylate (to achieve the molar ratio RB=0.0069) and 0.17 g of dilauroyl peroxide are again added to the heterogeneous reaction medium. The medium obtained is maintained at 80° C. with mechanical stirring for another three hours. It is then cooled to 35° C. and filtered on paper and the precipitate collected is finally dried in an oven at 50° C. under a vacuum of 20 mbar. 3.32 g of the copolymer (P3) are thus isolated, which corresponds to a final yield of 27%.

Preparation of a Tulipalin A-Itaconic Acid Copolymer (RA=1), Crosslinked with Pentaerythritol Tetraacrylate (RB=0.004) According to the Invention (P4)

50.0 g of demineralized water are poured into a reactor with mechanical stirring. 6.50 g (0.05 mol) of itaconic acid, 1.39 g of a 48 wt % (0.00167 mol) sodium hydroxide solution to partially dissolve the itaconic acid, 4.91 g (0.05 mol) of Tulipalin A and 0.1057 g (0.0003 mol) of pentaerythritol tetraacrylate are added thereto. The dispersion obtained is deoxygenated by nitrogen sparging for 40 minutes and then heated to 80° C. 0.1356 g of 2,2′-azobis(2-methylpropanamidine) is then added thereto and the whole medium is maintained at 80° C. with mechanical stirring for three hours. It is then cooled to 35° C. and filtered on paper and the precipitate collected is finally dried in an oven at 50° C. under a vacuum of 20 mbar. 4.77 g of the copolymer (P4) are thus isolated, which corresponds to a final yield of 41.4%.

Preparation of a Tulipalin A-Itaconic Acid Copolymer (RA=2), Crosslinked with Trimethylolpropane Triacrylate (RB=0.004) According to the Invention (P5)

30.0 g of tert-butanol and 1.5 g of demineralized water are poured into a reactor with mechanical stirring. 6.50 g (0.05 mol) of itaconic acid, 4.91 g (0.05 mol) of Tulipalin A and 0.1057 g (0.0003 mol) of trimethylolpropane triacrylate are then added thereto. The dispersion obtained is deoxygenated by nitrogen sparging for 45 minutes and then heated to 80° C. 0.20 g of dilauroyl peroxide is then added thereto and the whole medium is maintained at 80° C. with mechanical stirring for three hours. 4.91 g (0.05 mol) of Tulipalin A are then added (to achieve the molar ratio RA=2), followed by 0.1057 g (0.0003 mol) of trimethylolpropane triacrylate (to achieve the molar ratio RB=0.004) and 0.20 g of dilauroyl peroxide are again added to the heterogeneous reaction medium. The medium obtained is maintained at 80° C. with mechanical stirring for another three hours. It is then cooled to 35° C. and filtered on paper and the precipitate collected is finally dried in an oven at 50° C. under a vacuum of 20 mbar. 13.01 g of the copolymer (P5) are thus isolated, which corresponds to a final yield of 79%.

### Preparation of a Tulipalin A-Itaconic Acid Copolymer (RA=2) According to the Invention (P6)

30.0 g of tert-butanol and 1.5 g of demineralized water are poured into a reactor with mechanical stirring. 6.50 g (0.05 mol) of itaconic acid and 4.91 g (0.05 mol) of Tulipalin A are then added thereto. The dispersion obtained is deoxygenated by nitrogen sparging for 50 minutes and then heated to 80° C. 0.20 g of dilauroyl peroxide is then added thereto and the whole medium is maintained at 80° C. with mechanical stirring for three hours. 4.91 g (0.05 mol) of Tulipalin A are then added (to achieve the molar ratio RA=2) and 0.20 g of dilauroyl peroxide are again added to the heterogeneous reaction medium. The medium obtained is maintained at 80° C. with mechanical stirring for another three hours. It is then cooled to 35° C. and filtered on paper and the precipitate collected is finally dried in an oven at 50° C. under a vacuum of 20 mbar. 12.36 g of the copolymer (P6) are thus isolated, which corresponds to a final yield of 75%.

Preparation of a Tulipalin a (Ia)-Itaconic Acid Copolymer (RA=2) Carried Out with Recycling of the Filtrate According to the Invention (P7)

16.0 g of the filtrate from a test of copolymerization of Tuliplain A and itaconic acid (RA=1; with addition of the Tulipalin in one go) in tert-butanol are placed in the reactor (this filtrate containing itaconic acid which has not reacted with the Tulipalin A during a previous synthesis). 2.45 g (0.025 mol) of Tulipalin A are added. The dispersion obtained is deoxygenated by nitrogen sparging for 50 minutes and then heated to 80° C. 0.10 g of dilauroyl peroxide is then added thereto and the whole medium is maintained at 80° C. with mechanical stirring for 6 hours. The reaction medium is then cooled to 35° C. and filtered on paper and the precipitate collected is finally dried in an oven at 50° C. under a vacuum of 20 mbar. 2.80 g of copolymer P7) are isolated, corresponding to a final yield of 48.6%.

Preparation of a Tulipalin A-Itaconic Acid Copolymer (RA=1), Crosslinked with Pentaerythritol Tetraacrylate (RB=0.003) According to the Invention (P8)

30.0 g of tert-butanol and 1.5 g of demineralized water are poured into a reactor with mechanical stirring. 6.5 g (0.05 mol) of itaconic acid, 4.9 g (0.05 mol) of Tulipalin A and 0.11 g (0.0003 mol) of pentaerythritol tetraacrylate are added thereto. A homogeneous solution is obtained. This solution is deoxygenated by nitrogen sparging for 45 minutes and then heated to 80° C. 0.2 g of dilauroyl peroxide is then added thereto and the whole medium is maintained at 80° C. with mechanical stirring for 3 hours. The reaction medium is then cooled to 35° C. and filtered on paper and the precipitate collected is finally dried in an oven at 50° C. under a vacuum of 20 mbar. 5.63 g of copolymer (P8) are isolated, corresponding to a final yield of 48.9%.

Preparation of a Tulipalin A-Itaconic Acid Copolymer (RA=1), Crosslinked with Pentaerythritol Tetraacrylate (RB=0.003) According to the Invention (P9)

50.0 g of tert-butanol and 1.5 g of demineralized water are poured into a reactor with mechanical stirring. 13.0 g (0.1 mol) of itaconic acid, 9.8 g (0.1 mol) of Tulipalin A (i.e. a molar ratio RA=1) and 0.21 g (0.0006 mol) of pentaerythritol tetraacrylate are added thereto. A suspension is obtained. This suspension is deoxygenated by nitrogen sparging for 45 minutes and then heated to 56° C. 0.4 g of dilauroyl peroxide is then added thereto and the whole medium is maintained at 56° C. with mechanical stirring for 3 hours. The reaction medium is then cooled to 25° C. and filtered on paper and the precipitate collected is finally dried in an oven at 50° C. under a vacuum of 20 mbar. 5.63 g of copolymer (P9) are isolated, corresponding to a final yield of 28.7%.

Preparation of a Tulipalin Homopolymer (Ia) Crosslinked with Pentaerythritol Tetraacrylate (P10) (Outside of the Invention)

30.0 g of tert-butanol and 1.5 g of demineralized water are poured into a reactor with mechanical stirring. 9.81 g (0.1 mol) of Tulipalin A and 0.1057 g (0.0003 mol) of pentaerythritol tetraacrylate are added thereto. A homogeneous solution is obtained. This solution is deoxygenated by nitrogen sparging for 60 minutes and then heated to 80° C. 0.20 g of dilauroyl peroxide is then added thereto and the whole medium is maintained at 80° C. with mechanical stirring for 3 hours. The reaction medium is then cooled to 35° C. and filtered on paper and the precipitate collected is finally dried in an oven at 50° C. under a vacuum of 20 mbar. 9.87 g of homopolymer (P10) are isolated. This polymer is not soluble (even after adding NaOH to adjust the pH to 6-8) in water and therefore does not exhibit thickening properties for aqueous phases.

Preparation Outside of the Invention of an Itaconic Acid Homopolymer (IIa) Crosslinked with Pentaerythritol Tetraacrylate (P11) (Outside the Invention)

The same process as that described for the homopolymer (P10) was carried out by replacing the 30.0 g of tert-butanol with 50.0 g of acetone and the 9.81 g of Tulipalin A with 13.0 g of itaconic acid. 13.2 g of homopolymer (P11) are isolated after drying the reaction medium, which corresponds to a yield of 100.0%. This polymer is soluble in water after adding sodium hydroxide to adjust the pH to 6-8 but does not exhibit thickening properties for aqueous phases.

Preparation of a Tulipalin A-Itaconic Acid Copolymer (Molar Ratio RA=0.2), Crosslinked with Pentaerythritol Tetraacrylate (Molar Ratio RB=0.0055) (P12) (Outside the Invention)

60.0 g of tert-butanol and 3.05 g of demineralized water are poured into a reactor with mechanical stirring. 21.68 g (0.16667 mol) of itaconic acid, 1.63 g (0.01667 mol) of Tulipalin A and 0.194 g (0.00055 mol) of pentaerythritol tetraacrylate are then added thereto. The dispersion obtained is deoxygenated by nitrogen sparging for 45 minutes and then heated to 80° C. 0.365 g of dilauroyl peroxide is then added thereto and the whole medium is maintained at 80° C. with mechanical stirring for three hours. 1.63 g (0.01667 mol) of Tulipalin A are then added (to achieve the molar ratio RA=0.2), followed by 0.194 g (0.00055 mol) of pentaerythritol tetraacrylate (to achieve the molar ratio RB=0.0055) and 0.365 g of dilauroyl peroxide are again added to the heterogeneous reaction medium. The medium obtained is maintained at 80° C. with mechanical stirring for another three hours. It is then cooled to 35° C. and filtered on paper and the precipitate collected is finally dried in an oven at 50° C. under a vacuum of 20 mbar. 3.91 g of the copolymer (P5) are thus isolated, which corresponds to a final yield of 15.4%.

Preparation of a Tulipalin A-Itaconic Acid Copolymer (IIa) (Molar Ratio RA=4), Crosslinked with Pentaerythritol Tetraacrylate (Molar Ratio RB=0.0036) (P13) (Outside the Invention)

30.0 g of tert-butanol and 1.5 g of demineralized water are poured into a reactor with mechanical stirring. 2.60 g (0.02 mol) of itaconic acid, 3.92 g (0.04 mol) of Tulipalin A and 0.063 g (0.00018 mol) of pentaerythritol tetraacrylate are then added thereto. A dispersion is obtained. The dispersion obtained is deoxygenated by nitrogen sparging for fifty minutes and then heated to 80° C. 0.12 g of dilauroyl peroxide is then added thereto and the whole medium is maintained at 80° C. with mechanical stirring for three hours. 3.92 g (0.04 mol) of Tulipalin A are then added (to achieve the molar ratio RA=4) followed by 0.063 g (0.00018 mol) of pentaerythritol tetraacrylate (to achieve the molar ratio RB=0.0040) and 0.12 g of dilauroyl peroxide. The medium obtained is maintained at 80° C. with mechanical stirring for another three hours. It is then cooled to 35° C. and filtered on paper and the precipitate collected is finally dried in an oven at 50° C. under a vacuum of 20 mbar. 8.9 g of the expected copolymer (P13) are thus isolated, which corresponds to a final yield of 84%.
Evaluation of the thickening properties of the polymers prepared. The thickening properties of copolymers based on Tulipalin A and itaconic acid according to the invention (P1), (P2), (P3), (P4), (P5), (P6), (P7), (P8) and (P9) are recorded in Table 1 below. They are compared with those of the polymers outside the invention (P10), (P11), (P12) and (P13). Gels are prepared by dispersing the polymer in water and by adjusting the pH to a value greater than or equal to 6 and less than or equal to 8 with sodium hydroxide if necessary.

TABLE 1
Viscosity of aqueous polymer gels (in mPa ·
s) Brookfield LVT (spindle 4; speed 6 rpm)

| Polymer | | | |
|---|---|---|---|
| according | Weight proportions of | Without | |
| to the | polymer in the gel | NaCl | With 01% NaCl |
| P1 | 2% | 14.720 | 8.680 |
| P1 | 5% | >100 000 | Not determined (n.d.) |
| P2 | 2% | 8.840 | 2.560 |
| P2 | 5% | 63.000 | n.d. |
| P3 | 2% | 13.000 | 9.640 |
| P3 | 5% | >100 000 | 39.400 |
| P4 | 2% | 1.630 | n.d. |
| P4 | 5% | 10.000 | n.d. |
| P5 | 2% | 3.065 | 1.200 |
| P5 | 5% | 37.000 | n.d. |
| P6 | 2% | 1.220 | 860 |
| P6 | 5% | 11.600 | n.d. |
| P7 | 2% | 24.000 | n.d. |
| P7 | 5% | 580 | n.d. |
| P8 | 5% | 43.800 | n.d. |
| P9 | 5% | 34.000 | n.d. |
| Polymer outside | Weight proportions of | | |
| P10 | | n.d. | n.d. |
| P11 | | Non-thickening | n.d. |
| P12 | 2% | 4.040 | 3.180 |
| P12 | 5% | 41.600 | n.d. |
| P13 | 2% | 5 | 5 |
| P13 | 5% | 20 | n.d. |

Examples of cosmetic topical formulations according to the invention

TABLE 2
AHA foaming formulation

| Ingredients | Weight proportions |
|---|---|
| Demineralized water | qs 100.0% |
| Glycerol | 2.0% |
| Copolymer (P1) | 1.0% |
| ORAMIX ™ CG110 | 5.0% |
| Sodium lauryl ether sulfate 28% | 6.0% |
| Glycolic acid 70% | 5.0% |
| EUXYL ™ K712 | 1.0% |
| Triethanolamine | qs pH 4 |

TABLE 3
Sun protection formulation

| Ingredients | Weight proportions |
|---|---|
| Demineralized water | qs 100.00% |
| Copolymer (P1) | 0.50% |
| FLUIDIFEEL ™ EASY | 3.00% |
| LANOL ™ 37T | 8.00% |
| EMOGREEN ™ L19 | 4.00% |
| NEO HELIOPAN ™ OS | 5.00% |
| NEO HELIOPAN ™ BMT | 4.00% |
| NEO HELIOPAN ™ HMS | 5.00% |
| DL-alpha-Tocopherol | 0.05% |
| EUXYL ™ K903 | 1.00% |
| CONTACTICEL ™ | 2.00% |
| EPHEMER ™ | 2.00% |
| TINOSORB ™ M | 5.00% |
| Citric acid 25% | 0.04% |

TABLE 4
Sun protection formulation SPF 50

| Ingredients | Weight proportions |
|---|---|
| Demineralized water | qs 100.0% |
| Glycerol | 3.0% |
| Copolymer (P3) | 0.5% |
| NEO HELIOPAN ™ HYDRO | 1.5% |
| Triethanolamine 50% | 1.7% |
| SENSANOV ™ WR | 3.0% |
| NEO HELIOPAN ™ OS | 5.0% |
| NEO HELIOPAN ™ BMT | 6.0% |
| LANOL ™ 37T | 7.0% |
| DUB ™ SSIC | 5.0% |
| A15-TiO2-SX-NJE8 | 5.0% |
| DL-alpha-Tocopherol | 0.2% |
| EUXYL ™ PE9010 | 0.5% |
| SENSIVA ™ PA40 | 0.5% |

TABLE 5
Oil-in-water emulsions

| | |
|---|---|
| Ingredients | Weight proportions |

| MONTANOV ™ 68 | 1.0% | 1.0% | 1.0% |
|---|---|---|---|
| TRIGLYCERIDES 5545 | 20.0% | 20.0% | 20.0% |
| Demineralized water | qs 100.0% | | |
| Copolymer (P1) | 0.3% | 0.5% | 1.0% |
| EUXYL ™ PE9010 | 1.0% | 1.0% | 1.0% |

TABLE 6
Oil-in-water emulsions with emollients

| | |
|---|---|
| Ingredients | Weight proportions |

| MONTANOV 68 | 3.0% | 3.0% | 3.0% |
|---|---|---|---|
| TRIGLYCERIDES 5545 | 20.0% | 20.0% | 25.0% |
| EMOGREEN ™ L19 | 5.0% | 10.0% | 0.0% |
| EMOGREEN HP40 | 0.0% | 0.0% | 5.0% |
| Demineralized water | qs 100.0% | | |
| Copolymer (P1) | 1.0% | 0.5% | 1.0% |
| EUXYL PE9010 | 1.0% | 1.0% | 1.0% |

TABLE 7
Oil-in-water emulsions with plant oils

| Ingredients | Weight proportions | | | |
|---|---|---|---|---|
| MONTANOV ™ 68 | 3.0% | 3.0% | 3.0% | 3.0% |
| LANOL ™ 2681 | 0.0% | 0.0% | 0.0% | 0.0% |
| LANOL ™ 99 | 20.0% | 0.0% | 0.0% | 0.0% |
| Sweet almond oil | 0.0% | 30.0% | 0.0% | 0.0% |
| Jojoba oil | 0.0% | 0.0% | 30.0% | 0.0% |
| Olive oil | 0.0% | 0.0% | 0.0% | 30.0% |
| Demineralized water | qs 100.0% | | | |
| Copolymer (P5) | 0.5% | 1.0% | 1.0% | 1.0% |
| EUXYL ™ PE9010 | 1.0% | 1.0% | 1.0% | 1.0% |

TABLE 8
Oil-in-water emulsions

| Ingredients | Amount by weight (%) | | | |
|---|---|---|---|---|
| FLUIDIFEEL ™ EASY | 3.0% | 3.0% | 3.0% | 3.0% |
| TRIGLYCERIDES 5545 | 20.0% | 20.0% | 10.0% | 10.0% |
| EMOGREEN ™ L15 | 0.0% | 0.0% | 5.0% | 5.0% |
| Demineralized water | qs 100.0% | | | |
| Copolymer (P5) | 1.0% | 0.5% | 0.5% | 1.0% |
| EUXYL ™ PE9010 | 1.0% | 1.0% | 1.0% | 1.0% |

TABLE 9
Oil-in-water emulsions

| | |
|---|---|
| Ingredients | Weight proportions |

| FLUIDIFEEL ™ EASY | 1.0% | 1.0% | 1.0% |
|---|---|---|---|
| TRIGLYCERIDES 5545 | 20.0% | 10.0% | 0.0% |
| EMOGREEN ™ L15 | 0.0% | 5.0% | 0.0% |
| LANOL ™ 2681 | 0.0% | 0.0% | 15.0% |
| Demineralized water | qs 100.0% | | |
| Copolymer (P2) | 0.5% | 0.5% | 1.0% |
| EUXYL ™ PE9010 | 1.0% | 1.0% | 1.0% |

TABLE 10
Moisturizing and anti-ageing oil-in-water emulsion

| Ingredients | Weight proportions |
|---|---|
| Demineralized water | qs 100.0% |
| Copolymer (P1) | 1.0% |
| FLUIDIFEEL ™ EASY | 3.0% |
| Sweet almond oil | 10.0% |
| LANOL ™ 2681 | 7.0% |
| DUB ZENOAT ™ | 3.0% |
| AQUAXYL ™ | 3.0% |
| APAR'AGE ™ | 2.0% |
| EUXYL ™ K903 | 1.0% |
| Fragrance | 0.1% |
| Lactic acid | qs pH = 5 |

TABLE 13
Ingredients used in the abovementioned topical formulations

| Trade name | Composition or INCI name |
|---|---|
| ORAMIX CG110 | Caprylyl-Capryl Glucoside |
| EUXYL K712 | Sodium Benzoate (and) Potassium Sorbate (and) |
| | Aqua |
| FLUIDIFEEL ™ | Lauryl Glucoside (and) Myristyl Glucoside (and) |
| EASY | |
| LANOL ™ 37T | Triheptanoin |
| EMOGREEN ™ L19 | C15-19 Alkane |
| NEO HELIOPAN ™ | Ethylhexyl salicylate |
| OS | |
| NEO HELIOPAN ™ | Bis-Ethylhexyloxyphenol Methoxyphenyl |
| BMT | Triazine |
| NEO HELIOPAN ™ | Homosalate |
| HMS | |
| EUXYL ™ K903 | Benzyl Alcohol (and) Benzoic Acid (and) |
| CONTACTICEL ™ | Aqua (and) Butylene Glycol (and) Hydrolyzed |
| EPHEMER ™ | Caprylic/Capric Triglyceride - Undaria Pinnatifida |
| TINOSORB ™ M | Methylene Bis-Benzotriazolyl |
| | Tetramethylbutylphenol |
| NEO HELIOPAN ™ | Phenylbenzimidazole Sulfonic Acid |
| HYDRO | |
| SENSANOV ™ WR | C20-22 Alkyl Phosphate (and) C20-22 Alcohols |
| DUB SSIC ™ | Isocetyl Stearoyl Stearate |
| A15-TiO2-SX-NJE8 | Titanium Dioxide (and) Silica (and) Jojoba Esters |
| EUXYL ™ PE9010 | Phenoxyethanol (and) Ethylhexylglycerin |
| SENSIVA ™ PA40 | Phenylpropanol (and) Propanediol (and) Caprylyl |
| MONTANOV ™ 68 | Cetearyl Alcohol (and) Cetearyl Glucoside |
| TRIGLYCERIDES | Caprylic-Capric Triglyceride |
| 5545 | |
| EMOGREEN ™ | C15-19 Alkane (and) Hydrogenated |
| HP40 | Polyfarnesene |
| LANOL ™ 2681 | Coco-Caprylate-Caprate |
| LANOL ™ 99 | Isononyl Isononanoate |
| Sweet almond oil | Prunus amygdalus dulcis oil |
| Jojoba oil | Simmondsia chinensis seed oil |
| Olive oil | Olea europaea fruit oil |
| EMOGREEN ™ L15 | C15-19 Alkane |
| DUB ZENOAT ™ | Propanediol Dicaprylate |
| AQUAXYL ™ | Xylitylglucoside (and) Anhydroxylitol (and) |
| | Xylitol |
| APAR'AGE ™ | Water (and) Propanediol (and) Asparagopsis |
| | Armata |

1. A linear or crosslinked copolymer consisting of monomer units derived from 3-methylenedihydro-2(3H)-furanone of formula (I): 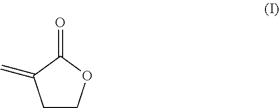
and of monomer units derived from methylene-1,4-butanedioic acid of formula (II): 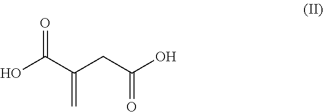
wherein a molar ratio RA, having as numerator a number of moles of monomer units derived from the compound of formula (I) and as denominator a number of moles of monomer units derived from the compound of formula (II), that is greater than or equal to 0.4 and less than or equal to 3.0.

2. The linear or crosslinked copolymer according to claim 1, wherein the molar ratio RA is greater than or equal to 1.5 and less than or equal to 2.5.

3. The linear or crosslinked copolymer according to claim 1, wherein the copolymer is the crosslinked copolymer, and wherein a molar ratio RB, having as numerator a number of moles of monomer units derived from a crosslinking monomer and as denominator a sum of the number of moles of monomer units derived from the compound of formula (I) and of moles of monomer units derived from the compound of formula (II), that is greater than or equal to 0.0010 and less than or equal to 0.0070.

4. The linear or crosslinked copolymer according to claim 3, wherein the molar ratio RB is greater than or equal to 0.0020 and less than or equal to 0.0060.

5. The linear or crosslinked copolymer according to claim 3, wherein the monomer units derived from the crosslinking monomer are from a compound selected from the group consisting of methylenebis(acrylamide), pentaerythritol tetraacrylate, trimethylolpropane triacrylate, triallylamine, pentaerythritol triallyl ether, 1,6-hexanediol diacrylate, ethylene glycol diacrylate, sodium diallyloxyacetate (DAOAS), and combinations thereof.

6. The linear or crosslinked copolymer according to claim 5, wherein the monomer units derived from the crosslinking monomer are from a compound selected from the group consisting of pentaerythritol tetraacrylate, trimethylolpropane triacrylate, and combinations thereof.

7. A process for preparing the linear or crosslinked copolymer as defined in claim 1, the process comprising:
[Chem 01] a) providing:
a solvent selected from the group comprising water, ethanol, propanol, isopropanol, butanol, isobutanol, sec-butanol, tert-butanol, acetone, methyl ethyl ketone, cyclohexane, ethyl acetate, or a mixture of two or more thereof,
the monomer of formula (II) and the monomer of formula (I) in molar proportions such that a molar ratio R′A, having as numerator a number of moles of compound of formula (I) and as denominator a number of moles of compound of formula (II), is greater than or equal to 0.4 and less than or equal to 3.0; and
optionally, the crosslinking monomer is selected from the group consisting of methylenebis(acrylamide), pentaerythritol tetraacrylate, trimethylolpropane triacrylate, triallylamine, pentaerythritol triallyl ether, 1,6-hexanediol diacrylate, ethylene glycol diacrylate, sodium diallyloxyacetate, and combinations thereof, in molar proportions such that a molar ratio R′B, having as numerator a number of moles of crosslinking monomer and as denominator a sum of the numbers of moles of compound of formula (I) and of compound of formula (II), is greater than or equal to 0.001 and less than or equal to 0.007;
[Chem 02] b) adding all of the moles of compound of formula (II) to the solvent provided in step a) to form a solution or dispersion of the compound of formula (II),
[Chem 03] optionally, c) partially or completely neutralizing the compound of formula (II), by adding a base to the solution or dispersion formed in step b), to form a solution or dispersion of the partially or completely neutralized compound of formula (II);
[Chem 04] d) adding either part or all of the moles of compound of formula (I) provided in step a) to the solution or dispersion formed in step b) or to the solution or dispersion formed in step c), to form a solution or dispersion of the compound of formula (II), optionally partially or completely neutralized, and of the compound of formula (I);
[Chem 05] optionally, e) adding either part or all of the moles of crosslinking monomer to the solution or dispersion obtained in step d), to form a solution or dispersion of the compound of formula (II), optionally partially or completely neutralized, of the compound of formula (I) and of the crosslinking monomer;
[Chem 06] f) deoxygenating the solution or dispersion obtained in step d), or in optional step e) respectively, by sparging an inert gas through it for a period of greater than or equal to 30 minutes and less than or equal to 90 minutes, to obtain a deoxygenated solution or dispersion of the compound of formula (II), optionally partially or completely neutralized, of the compound of formula (I) and of the crosslinking monomer;
[Chem 07] g) heating the solution or dispersion obtained in step f) at a temperature above or equal to 50° C. and below or equal to 95° C., to obtain a deoxygenated and heated solution or dispersion of the compound of formula (II), optionally partially or completely neutralized, of the compound of formula (I) and of the crosslinking monomer;
[Chem 08] h) polymerizing the monomers present in the deoxygenated and heated solution or dispersion obtained in step g), by adding a polymerization initiator and maintaining at a polymerization temperature above or equal to 50° C. and below or equal to 95° C., for a period greater than or equal to 150 minutes and less than or equal to 300 minutes, to obtain a precipitate of an optionally crosslinked copolymer and optionally of the unreacted monomer of formula (II) in the solvent provided in step a);
[Chem 09] optionally, i) adding to the solvent obtained in step h), comprising the precipitate of the optionally crosslinked copolymer and optionally the unreacted monomer of formula (II), the remainder of the compound of formula (I) and optionally the remainder of the crosslinking monomer provided in step a), and then polymerizing the remainder of the monomers present by adding a polymerization initiator while maintaining the temperature at a value above or equal to 50° C. and below or equal to 95° C., for a period greater than or equal to 150 minutes and less than or equal to 300 minutes, to obtain a dispersion of the optionally crosslinked copolymer;
[Chem 10] j) cooling the dispersion of the optionally crosslinked copolymer obtained in step i) to a temperature above or equal to 15° C. and below or equal to 30° C.; and
[Chem 11] either k) filtering the cooled dispersion obtained in step j) to collect the optionally crosslinked copolymer, followed by 1) drying the collected optionally crosslinked copolymer,
[Chem 12] or m) spraying the cooled dispersion obtained in step j), in order to obtain the optionally crosslinked copolymer in powder form.

8. The preparation process according to claim 7, wherein step b), optional step c), step d) and optional step e) are simultaneous and constitute a step B).

9. The preparation process according to claim 7, wherein the solvent provided in step a) is either water, tert-butanol or a tert-butanol/water mixture.

10. The preparation process according to claim 9, comprising the following successive steps:
a) providing:
a tert-butanol/water mixture comprising, per 100% by weight, a weight proportion of greater than or equal to 90% and less than or equal to 98% by weight of tert-butanol and a weight proportion of greater than or equal to 2% by weight and less than or equal to 10% by weight of water;
the monomer of formula (II) and the monomer of formula (I) in molar proportions such that the molar ratio R′A, having as numerator the number of moles of compound of formula (I) and as denominator the number of moles of compound of formula (II), is greater than or equal to 1.0 and less than or equal to 3.0; and optionally
the crosslinking monomer selected from the group consisting of pentaerythritol tetraacrylate, trimethylol propane triacrylate, and combinations thereof in molar proportions such that the molar ratio R′B, having as numerator the number of moles of crosslinking monomer and as denominator the sum of the numbers of moles of compound of formula (I) and of compound of formula (II), is greater than or equal to 0.003 and less than or equal to 0.005;
B) adding, to the tert-butanol/water mixture provided in step a), all of the moles of the compound of formula (II), 50% of the moles of compound of formula (I), and optionally 50% of the moles of the crosslinking monomer to form a solution or a dispersion of the compound of formula (II), of the compound of formula (I) and optionally of the crosslinking monomer;
f) deoxygenating the solution or dispersion obtained in step B), by sparging nitrogen through it for a period greater than or equal to 45 minutes and less than or equal to 60 minutes, to obtain a deoxygenated solution or dispersion of the compound of formula (II), of the compound of formula (I) and optionally of the crosslinking monomer;
g) heating the solution or dispersion obtained in step f) at a temperature above or equal to 75° C. and below or equal to 85° C., to obtain a deoxygenated and heated solution or dispersion of the compound of formula (II), of the compound of formula (I) and optionally of the crosslinking monomer;
h) polymerizing the monomers present in the deoxygenated and heated solution or dispersion obtained in step g), by adding dilauroyl peroxide and maintaining at a polymerization temperature above or equal to 75° C. and below or equal to 85° C., for a period greater than or equal to 150 minutes and less than or equal to 200 minutes, to obtain a precipitate of the optionally crosslinked copolymer and optionally of the unreacted monomer of formula (II) in the solvent provided in step a); and
i) adding to the solvent obtained in step h), comprising the precipitate of the optionally crosslinked copolymer and optionally the unreacted monomer of formula (II), the remainder of the compound of formula (I) and optionally the remainder of the crosslinking monomer provided in step a), and then polymerizing the remainder of the monomers present by adding dilauroyl peroxide and maintaining at a polymerization temperature above or equal to 50° C. and below or equal to 85° C., for a period greater than or equal to 150 minutes and less than or equal to 200 minutes, to obtain a dispersion of the optionally crosslinked copolymer;
j) cooling the dispersion of the optionally crosslinked copolymer obtained in step i) to a temperature above or equal to 15° C. and below or equal to 30° C.; and
either k) filtering the cooled dispersion obtained in step j) to collect the optionally crosslinked copolymer, followed by a step 1) of drying the collected optionally crosslinked copolymer,
or m) spraying the cooled dispersion obtained in step j), in order to obtain the optionally crosslinked copolymer in powder form.

11. A method, for thickening, stabilizing or emulsifying a cosmetic or topical pharmaceutical formulation or for suspending solid particles therein, the method comprising providing the linear or crosslinked copolymer as defined in claim 1 to the cosmetic or topical pharmaceutical formulation.

12. A cosmetic or topical pharmaceutical formulation comprising, per 100.0% of its weight, from 0.1% to 10.0% of the linear or crosslinked copolymer as defined claim 1, as a thickener, as a stabilizer or as an emulsifier of the cosmetic or pharmaceutical topical formulation or as agent capable of suspending solid particles within the cosmetic or pharmaceutical topical formulation.

13. The process according to claim 7, wherein the deoxygenating comprises sparging nitrogen.

14. The process according to claim 7, wherein the polymerization initiator is dilauroyl peroxide or 2,2′-azobis(2-methylpropionamidine) dihydrochloride.

15. The cosmetic or topical pharmaceutical formulation of claim 12, wherein the formulation comprises, per 100.0% of its weight, from 0.5% to 5.0% of the linear or crosslinked copolymer as defined claim 1.
